(19) United States
(12) Patent Application Publication
(10) Pub. No.: US 2022/0288418 A1
(43) Pub. Date: Sep. 15, 2022

(54) POST-ABLATIVE MODULATION OF RADIATION THERAPY

(71) Applicant: Montefiore Medical Center, Bronx, NY (US)

(72) Inventors: Chandan GUHA, Scarsdale, NY (US); Talicia SAVAGE, New York, NY (US); Sanjay PANDEY, New York, NY (US)

(21) Appl. No.: 17/635,985

(22) PCT Filed: Aug. 26, 2020
(86) PCT No.: PCT/US2020/048020

§ 371 Date: Feb. 16, 2022

Related U.S. Application Data
(60) Provisional application No. 62/892,273, filed on Aug. 27, 2019.

(51) Int. Cl. A61N 5/10

(52) U.S. Cl. CPC A61N 5/1038

(57) ABSTRACT

Methods and systems are provided for treating a cancer in a subject, the method comprising providing an ablative dose of radiation therapy to a first region comprising the cancer followed by a sub-ablative dose to a second region, wherein the sub-ablative dose is administered within 1 hour to 4 days after the ablative dose.

### CROSS REFERENCE

This application claims the benefit of U.S. Provisional Application No. 62/892,273 filed on Aug. 27, 2019, which is incorporated by referenced herein in its entirety.

### STATEMENT AS TO FEDERALLY SPONSORED RESEARCH

This invention was made with government support under R01CA22686 (CG),1S100D019961-01 and 1S10RR029545-01, awarded by National Institutes of Health. The government has certain rights in the invention.

### BACKGROUND OF THE INVENTION

Cancer is a leading cause of death worldwide and is currently the second leading cause of death in the United States. Conventional treatment options for tumors include cytotoxic therapies such as radiation therapy (RT) and chemotherapy. Local tumor control by surgery or RT can fail because of eventual metastatic progression, even in the presence of systemic therapy. More recently immunotherapies have been proposed as possible treatment options to inhibit metastatic progression, both alone and in combination with other therapies. Immunotherapies include check-point inhibitors, tumor vaccines and adoptive cell transfer. Although immunotherapies have shown promise in the treatment of some solid tumors, such as melanoma and renal-cell carcinoma, immunotherapies have been less successful in other more fibrotic tumors such as pancreatic cancer. Even within cancer types, there are subsets of responders and non-responders, such that many people are less than ideally treated.

Although RT can be used to treat cancers, the cytotoxic effect of RT can limit its usefulness. For example, RT of tumors located on an organ can substantially damage the organ where the tumors are located. Metastatic tumors can be situated at locations remote from the primary tumor, such that the primary and metastatic tumors are located on more than one organ. Due to cytotoxic effects, RT of more than one organ can lead to damage to more than one organ in at least some instances.

In light of the above, it would be desirable to have improved methods and apparatus of treating cancer. Ideally, these methods and apparatus would inhibit metastatic progression of cancer and allow treatment of tumors on delicate organs and tissue structures with causing damage.

### SUMMARY OF THE INVENTION

The present methods and apparatus provide improved treatment of cancer with RT. A tumor can be treated with an ablative amount of radiation, and a subsequent sub-ablative treatment at the same or a different location. The subsequent sub-ablative treatment can be applied within a time from about 1 hour to about 4 days from the ablative treatment, in order provide an adaptive immune response to antigens on the cancer cells. The subsequent sub-ablative treatment can be applied at many locations, such as the same location on the tumor receiving the ablative dose, another tumor, or locations susceptible to metastasis. In some embodiments, the time between the ablative treatment and the sub-ablative treatment allows for structural changes to the tumor microenvironment, including the vasculature of the tumor. Because the subsequent treatment is sub-ablative, the subsequent treatment can be applied at many locations, even locations without an identified tumor, and the subsequent treatment may comprise a whole body treatment, for example. The radiotherapy can be administered in many ways, such as with a radiotherapy machine or brachytherapy and combinations thereof.

While the treatment can be performed in many ways, in some embodiments an initial ablative radiotherapy treatment generates a significant number of antigens, which are presented to the immune system. An adaptive immune response is generated in response to the antigens presented from the first treatment. A subsequent sub-ablative treatment can affect the tumor microenvironment and increase the extent to which immune cells can enter the tumor. The subsequent sub-ablative treatment can result in increased perfusion of the tumor (e.g. “cracking” of the tumor) so as to modify the tumor microenvironment, and can result in other changes to the tumor microenvironment that promotes an immune response. Although reference is made to treating a tumor with a sub-ablative dose of RT, tissues susceptible to tumors can be prophylactically treated with RT to inhibit tumor growth in those tissues.

In some embodiments, described herein is a method of treating a cancer in a subject, the method comprising providing an ablative dose of radiation therapy to a first region comprising the cancer followed by a sub-ablative dose to a second region, wherein the sub-ablative dose is administered after the ablative dose. In certain embodiments, the sub-ablative dose is administered at least 1 hour after the ablative dose. In certain embodiments, the sub-ablative dose is administered at least 1 day after the ablative dose. In certain embodiments, the sub-ablative dose is administered no more than 4 days after the ablative dose.

In some embodiments, disclosed herein is a method of treating a cancer in a subject, the method comprising providing an ablative dose of radiation therapy to a first region comprising the cancer followed by a sub-ablative dose to a second region, wherein the sub-ablative dose is administered within 1 hour to 4 days after the ablative dose. In some embodiments, a cumulative amount of radio therapy delivered to the second region throughout a course of treatment comprises less than an ablative dose. In some embodiments, the cumulative amount less than an ablative dose comprises a plurality of sub-ablative doses. In some embodiments, the first region comprises a region of a tumor and optionally the second region comprises the region of the tumor. In some embodiments, the first region comprises a region of a first tumor and the second region comprises a region of a second tumor. In some embodiments, the first tumor comprises a primary tumor and the second tumor comprises a metastatic tumor. In some embodiments, the first tumor comprises a metastatic tumor and the second tumor comprises a primary tumor. In some embodiments, the second region comprises a plurality of second regions and each of the plurality of second regions receives a cumulative amount of radiotherapy which is less than an ablative dose. In some embodiments, the second region comprises a different region from the first region. In some embodiments, the second region comprises a region of a tumor. In some embodiments, the second region comprises a region likely to develop a metastatic tumor and optionally the second region comprises a region of an organ selected from the group consisting of bones, lymph node, lung, liver, brain, adrenal gland, breast, eye, kidney, muscles, pancreas, salivary gland, and spleen. In some embodiments, the second region comprises a whole body of the subject scanned with the sub-ablative dose. In some embodiments, the first region comprises a region of a primary tumor of an organ selected from the group consisting of breast, bladder, brain, colon, rectal, endometrial, kidney, pancreas, prostate, liver, lung, skin, thyroid, uterus, lymph node, tonsil, thymus, spleen and bone marrow and the second region comprises a region of a metastatic tumor of an organ selected from the group consisting of bones, lymph node, lung, liver, brain, adrenal gland, breast, eye, kidney, muscles, pancreas, salivary gland, and spleen. In some embodiments, the first region comprises a region of a metastatic tumor of an organ selected from the group consisting of bone, lymph node, lung, liver, brain, adrenal gland, breast, eye, kidney, muscles, pancreas, salivary gland and spleen, and the second region comprises a primary tumor of an organ selected from the group consisting of breast, bladder, brain, colon, rectal, endometrial, kidney, pancreas, prostate, liver, lung, skin, thyroid, uterus, lymph node, tonsil, thymus, spleen and bone marrow. In some embodiments, the first region comprises an identified tumor and the second region does not comprise an identified tumor.

In some embodiments, disclosed herein is a computer readable medium configured with instructions that, when executed, cause a processor to provide instructions to a radiotherapy system to deliver an ablative dose of radiation therapy to a first region followed by delivery of a sub-ablative dose to a second region after the ablative dose. In some embodiments, the sub-ablative dose is delivered at least 1 hour after the ablative dose. In some embodiments, the sub-ablative dose is delivered at least 1 day after the ablative dose. In some embodiments, the sub-ablative dose is delivered no more than 4 days after the ablative dose.

In some embodiments, disclosed herein is a computer readable medium configured with instructions that, when executed, cause a processor to provide instructions to a radiotherapy system for an ablative dose of radiation therapy to a first region followed by a sub-ablative dose to a second region within 1 hour to 4 days after the ablative dose. In some embodiments, a cumulative amount of radio therapy delivered to the second region throughout a course of treatment comprises an amount of radio therapy which is less than an ablative dose. In some embodiments, the cumulative amount of radio therapy less than an ablative dose comprises a plurality of sub-ablative doses. In some embodiments, the first region comprises a region of a tumor and optionally the second region comprises the region of the tumor. In some embodiments, the first region comprises a region of a first tumor and the second region comprises a region of a second tumor. In some embodiments, the first tumor comprises a primary tumor and the second tumor comprises a metastatic tumor. In some embodiments, the first tumor comprises a metastatic tumor and the second tumor comprises a primary tumor. In some embodiments, the second region comprises a plurality of second regions and each of the plurality of second regions receives a cumulative amount of radiotherapy which is less than an ablative dose. In some embodiments, the second region comprises a different region from the first region. In some embodiments, the second region comprises a region of a tumor. In some embodiments, the second region comprises a region likely to develop a metastatic tumor and optionally the second region comprises a region of an organ selected from the group consisting of bones, lymph node, lung, liver, brain, adrenal gland, breast, eye, kidney, muscles, pancreas, salivary gland, and spleen. In some embodiments, the second region comprises a whole body of the subject scanned with the sub-ablative dose. In some embodiments, the first region comprises a region of a primary tumor of an organ selected from the group consisting of breast, bladder, brain, colon, rectal, endometrial, kidney, pancreas, prostate, liver, lung, skin, thyroid, uterus, lymph node, tonsil, thymus, spleen and bone marrow and the second region comprises a region of a metastatic tumor of an organ selected from the group consisting of bones, lymph node, lung, liver, brain, adrenal gland, breast, eye, kidney, muscles, pancreas, salivary gland, and spleen. In some embodiments, the first region comprises a region of a metastatic tumor of an organ selected from the group consisting of bone, lymph node, lung, liver, brain, adrenal gland, breast, eye, kidney, muscles, pancreas, salivary gland and spleen, and the second region comprises a primary tumor of an organ selected from the group consisting of breast, bladder, brain, colon, rectal, endometrial, kidney, pancreas, prostate, liver, lung, skin, thyroid, uterus, lymph node, tonsil, thymus, spleen and bone marrow. In some embodiments, the first region comprises an identified tumor and the second region does not comprise an identified tumor.

In some embodiments, disclosed herein is a radiotherapy system comprising a source of radiation to provide an ablative dose and a sub-ablative dose; and a processor coupled to the source of radiation, wherein the processor is configured with the instructions of described above. In some embodiments, the ablative dose comprises between 20 and 100 Gy at the first region. In some embodiments, the ablative dose comprises between 20 and 60 Gy at the first region. In some embodiments, the sub-ablative dose comprises between 0.1 and 2 Gy and optionally the sub-ablative dose comprises a plurality of sub-ablative doses and each of the plurality of sub-ablative doses comprises between 0.1 and 2 Gy at the second region. In some embodiments, the sub-ablative dose comprises between 0.1 and 0.5 Gy and optionally the sub-ablative dose comprises a plurality of sub-ablative doses and each of the plurality of sub-ablative dose/s comprises between 0.1 and 5 Gy at the second region. In some embodiments, three sub-ablative doses are administered. In some embodiments, more than three sub-ablative doses are administered. In some embodiments, a first sub-ablative dose is administered within 24 hours after the administration of the ablative dose. In some embodiments, a first sub-ablative dose is administered between 6 and 26 hours after the administration of the ablative dose. In some embodiments, the treatment reduces the size or intensity of the treated tumor as measured by imaging selected from the group consisting of a computed tomography scan, magnetic resonance imaging, positron emission tomography, a computed tomography scan. In some embodiments, the treatment increases the survival of the subject, reduces the number or severity of symptoms experienced by the subject, increases a number of immune cells in a microenvironment of the tumor, or increases a number of activated immune cells in a tumor microenvironment. In some embodiments, the radiation is selected from the group consisting of x-ray radiation, gamma ray radiation, alpha particle radiation, beta particle radiation, neutron particle radiation, external beam radiation and brachytherapy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a radiotherapy system suitable for use with the methods and programs in accordance with some embodiments of the present disclosure.

FIG. 2A illustrates a subject with a tumor in the liver as an example of a tumor in a first region, in accordance with some embodiments of the present disclosure.

FIG. 2B illustrates a subject to be treated in two regions that include a first region comprising a first tumor and a second region comprising a second tumor, in accordance with some embodiments of the present disclosure.

FIG. 3 illustrates examples of first, second and third treatment regions in a subject, in which the first treatment region comprises a tumor in the lung, the second treatment region comprises an entire lung, and the third treatment region comprises a metastatic tumor, in accordance with some embodiments of the present disclosure.

FIG. 4 illustrates a process for treating a patient in accordance with some embodiments of the present disclosure.

FIGS. 5A and 5B illustrate two models for radio-priming in accordance with some embodiments of the present disclosure. FIG. 5A illustrates a pre-priming treatment method wherein four sub-ablative doses are delivered to a tumor to prime the immune system prior to delivering an ablative dose. FIG. 5B illustrates a post-ablative modulation treatment method wherein an ablative dose of radiotherapy is delivered to a tumor followed by four sub-ablative doses, the initial ablative dose activates effector T cells and the subsequent sub-ablative doses alter the tumor microenvironment to increase immune infiltration, alter cytokine milieu and reprogram macrophages.

FIG. 6 illustrates a computer system for use in accordance with some embodiments of the present disclosure.

FIG. 7A illustrates a treatment scheme for tumor growth comparing pre and post priming with controls in 3LL tumor bearing mice. * denotes p<0.05 by Log Rank (Mantel-Cox). n=5 for FIGS. 7A-D.

FIG. 7B illustrates relative tumor growth of treatment groups.

FIG. 7C illustrates time to 3 times initial tumor volume.

FIG. 7D illustrates survival of treated mice.

FIG. 8A illustrates a schematic treatment scheme for local PAM and groups treated. n=4-5 mice.

FIG. 8B illustrates relative tumor growth curves for mice treated as in FIG. 8A. n=4-5 mice.

FIG. 8C illustrates time to three times initial volume of treated mice bearing 3LL tumors. n=28-35 mice.

FIG. 8D illustrates combined survival curve across multiple experiments in 3LL tumor bearing mice. * denotes p<0.05 Log Rank (Mantel-cox) and Grehan-Breslow-Wilcoxon tests.

FIG. 8E illustrates tumor growth of 3LL tumor bearing nude mice. n=10-13 mice.

FIG. 8F survival of 3LL tumor bearing nude mice. n=10-13 mice.

FIGS. 9A-9C relate to in vitro treatment of 3LL tumor cells. FIG. 9A illustrates a schematic of an in vitro treatment scheme for PAM on 3LL tumor cells. FIG. 9B illustrates cell death 6 and 24 hours after treatment as in FIG. 9A, as measured by LIVE/Dead fixable dye.

FIG. 9C illustrates immunomodulatory, stress and immunosuppressive markers phenotyped by surface expression at 6 hours and 12 hours after final treatment as in FIG. 9A. * p<0.05, ** p<0.005, *** p<0.0005, and **** p<0.0001, as determined by t-test.

FIGS. 9D-G relate to in vitro treatment of immune populations subsets with 0.5 Gy×4. FIG. 9D illustrates viability of sorted T cell subsets after treatment. FIG. 9E illustrates expression of CD25 and FOXP3 after treatment on sorted CD4+CD25+FOXP3+(Tregs). FIG. 9F illustrates expression of CD206, an M2 macrophage marker, on cytokine polarized bone marrow derived macrophages. FIG. 9G illustrates cytokine secretions of cytokine polarized bone marrow derived macrophages after treatment. * p<0.05, ** p<0.005, *** p<0.0005, and **** p<0.0001, as determined by t-test.

FIG. 10A illustrates a treatment and harvest schematic for local PAM treatment in 3LL tumor bearing mice.

FIG. 10B illustrates in vivo infiltration leukocytes and Tregs at days 6 and 10 after start of local PAM treatment by flow cytometry. * p<0.05, ** p<0.005 by ANOVA multiple comparison.

FIG. 10C illustrates whole tumor lysate RNA expression of FOXP3 at day 6. * p<0.05, ** p<0.005 by ANOVA multiple comparison.

FIG. 10D illustrates intratumoral infiltration of granzyme B secreting effector cells at day 6 and day 10 by flow cytometry.

FIG. 10E illustrates phenotyping for polarization of tumor-infiltrating macrophages at day 6 and day 10 after start of treatment by flow cytometry. * p<0.05, ** p<0.005 by ANOVA multiple comparison.

FIG. 11A illustrates lymphocyte percentages at day 6 and day 10 by flow cytometry in spleen. * p<0.05, ** p<0.005 by ANOVA multiple comparison all panels

FIG. 11B illustrates Lymphocyte percentages at day 6 and day 10 by flow cytometry in a draining lymph node. * p<0.05, ** p<0.005 by ANOVA multiple comparison all panels

FIG. 12A illustrates characterization of CD8 T cells and Treg populations in the draining lymph node at day 6 and day 10 after local PAM treatment. * p<0.05, ** p<0.005 t-test, n=4-6.

FIG. 12B illustrates characterization of CD8 T cells and Treg populations in the spleen at day 6 and day 10 after local PAM treatment. * p<0.05, ** p<0.005 t-test, n=4-6.

FIG. 12C illustrates ELISPOT of spleens from treated mice for functional cytokine secretion at day 6 and day 10 for granzyme B. * p<0.05, t-test, n=4-6.

FIG. 12D illustrates ELISPOT of spleens from treated mice for functional cytokine secretion at day 6 and day 10 for IFNγ. * p<0.05, t-test, n=4-6.

FIG. 13A illustrates tumor measurements for mice bearing orthotopic 4T1 tumors, treated with local PAM and controls. n=12-13 mice.

FIG. 13B illustrates relative tumor growth of 4T1 tumor bearing mice with local PAM. n=12-13 mice.

FIG. 13C illustrates survival curves of 4T1 mice treated with local PAM. n=12-13 mice.

FIG. 14A illustrates a treatment schematic for systemic PAM with whole lung irradiation after primary tumor ablation.

FIG. 14B illustrates survival at 2 months after inoculation and overall survival for systemic PAM treatments compared to primary tumor ablation alone. * denotes p<0.05 Log Rank (Mantel-cox) and Grehan-Breslow-Wilcoxon tests. n=26-27 mice

FIG. 14C illustrates India ink injected lungs 12 days with and without whole lung irradiation (Red arrows indicate macro-metastases) and graph of enumerated visible macro-metastases.

FIG. 14D illustrates histologic sections of lung (Red asterisks indicate metastatic lesions) and a graph of enumerated lesions.

FIG. 15A illustrates a PET scan 28 days after primary tumor ablation. Representative of 7 images. FIG. 15B illustrates a PET scan 28 days after primary tumor ablation and 12 days after whole lung irradiation. Representative of 6 images.

FIG. 16A illustrates Treg populations in whole lungs after whole lung PAM treatment. * p<0.05, ** p<0.005, ANOVA multiple comparison.

FIG. 16B illustrates whole lung phenotyping by flow cytometry 19 days after primary tumor ablation. * p<0.05 by ANOVA multiple comparison.

FIG. 16C illustrates characterization of GzB secreting T cells. * p<0.05, ** p<0.005, *** p<0.0005, ANOVA multiple comparison.

FIG. 16D illustrates histological staining of CD8 (brown) and FOXP3 (green) in metastatic lung lesions (22× scale).

FIG. 16E illustrates splenic CD45+ cells by flow cytometry 19 days after primary tumor ablation. * p<0.05, **** p<0.0001 by ANOVA multiple comparison all panels.

FIG. 16F illustrates splenic CD3+ T cells by flow cytometry 19 days after primary tumor ablation. * p<0.05, **** p<0.0001 by ANOVA multiple comparison all panels

FIG. 16G illustrates splenic monocytes and monocyte MHC Class II expression after treatment. * p<0.05, ** p<0.005, *** p<0.0005, **** p<0.0001 ANOVA multiple comparison.

FIG. 17 illustrates the effects of PAM-RT on local and systemic immunomodulation.

FIG. 18A illustrates survival of mice treated with no treatment, a single ablative dose or radiotherapy, pre-ablation priming or post-ablative modulation in accordance with some embodiments of the present disclosure.

FIG. 18B illustrates density of tumor vasculature in mice treated with no treatment, a single ablative dose or radiotherapy, post-ablative modulation or with four sub-ablative doses alone in accordance with some embodiments of the present disclosure.

FIG. 19A illustrates tumor growth in C57Bl6 mice treated with no treatment, a single ablative dose of radiotherapy, or with post-ablative modulation in accordance with some embodiments of the present disclosure.

FIG. 19B illustrates tumor growth in nude mice treated with no treatment, a single ablative dose of radiotherapy, or with post-ablative modulation in accordance with some embodiments of the present disclosure.

FIG. 20A illustrates survival in C57Bl6 mice treated with no treatment, a single ablative dose of radiotherapy, or with post-ablative modulation in accordance with some embodiments of the present disclosure.

FIG. 20B illustrates survival in nude mice treated with no treatment, a single ablative dose of radiotherapy, or with post-ablative modulation in accordance with some embodiments of the present disclosure.

FIG. 21A illustrates leukocyte infiltration in 3LL tumors of C57Bl6 mice treated with no treatment, a single ablative dose of radiotherapy (24 Gy), post-ablative modulation or pre-priming in accordance with some embodiments of the present disclosure. For the mice treated with a single ablative treatment leukocyte infiltration is shown one day, and five days, after treatment.

FIG. 21B illustrates leukocyte infiltration in 3LL tumors of C57Bl6 mice treated with no treatment, a single ablative dose of radiotherapy (24 Gy), post-ablative modulation or pre-priming in accordance with some embodiments of the present disclosure. For the mice treated with a single ablative treatment leukocyte infiltration is shown one day, and five days, after treatment.

FIG. 21C illustrates leukocyte infiltration in 4T1 tumors of C57Bl6 mice treated with no treatment, a single ablative dose of radiotherapy (24 Gy), post-ablative modulation or pre-priming treated in accordance with some embodiments of the present disclosure. For the mice treated with a single ablative treatment leukocyte infiltration is shown one day, and five days, after treatment.

FIG. 22A illustrates survival in mice treated with Trabectedin alone, a single ablative dose (24 Gy), post-ablative modulation (22 Gy+4×0.5 Gy), a single ablative dose and Trabectedin, or post-ablative modulation with Trabectedin in accordance with some embodiments of the present disclosure.

FIG. 22B illustrates tumor growth in mice treated with Trabectedin in accordance with some embodiments of the present disclosure.

FIG. 22C illustrates tumor growth in mice treated with a single ablative dose of radiotherapy and Trabectedin in accordance with some embodiments of the present disclosure.

FIG. 22D illustrates tumor growth in mice treated in with post-ablative modulation and Trabectedin accordance with some embodiments of the present disclosure.

FIG. 23 illustrates an experimental program in accordance with some embodiments of the present disclosure. Briefly, mice are injected with tumor cells (for example 4T1 cells) in the 4^{th} mammary fat pad, after about eight days the tumors are administered three ablative doses of radiotherapy over three days (3×20 Gy), and an anti-PD1 therapeutic or vehicle, after another twelve days some of the mice are treated with four doses of sub-ablative radiotherapy over four days (4×0.5 Gy), and treated with an anti-PD1 therapeutic or vehicle.

FIG. 24A illustrates survival in mice injected with D90 cells and treated with three ablative doses of radiotherapy, three ablative doses of radiotherapy and an anti-PD1 therapeutic, three ablative doses of radiotherapy and four sub-ablative doses, or three ablative doses of radiotherapy, four sub-ablative doses and an anti-PD1 therapeutic in accordance with some embodiments of the present disclosure.

FIG. 24B illustrates survival in mice injected intravenously with 4T1 treated an anti-PD1 therapeutic, four sub-ablative doses, or four sub-ablative doses and an anti-PD1 therapeutic in accordance with some embodiments of the present disclosure.

FIG. 25 illustrates survival in mice injected with 4T1 cells and treated with three ablative doses of radiotherapy, three ablative doses of radiotherapy and an anti-PD1 therapeutic, three ablative doses of radiotherapy and four sub-ablative doses, or three ablative doses of radiotherapy, four sub-ablative doses and an anti-PD1 therapeutic in accordance with some embodiments of the present disclosure.

FIG. 26 illustrates a significant decrease in Treg leading to a significantly increased CD8/Treg ratio in whole lungs after whole lung PAM treatment. * p<0.05, ANOVA multiple comparison.

### DETAILED DESCRIPTION OF THE INVENTION

The presently disclosed methods and apparatus can treat cancer with an ablative RT dose followed by a sub-ablative RT dose. The ablative RT dose can generate an adaptive immune response and the subsequent sub-ablative RT treatment can affect the tumor micro environment and may trigger production of antigenic material in cancer cells, which can result in an immunogenic response to the cancer cells. This sub-ablative does can be used to generate an immune response in delicate tissues in a manner that spares these delicate tissues such that these tissues remain viable subsequent to treatment. The presently disclosed methods and apparatus are well suited for combination with prior methods, compounds and apparatus for treating cancer. For example, the ablative and sub-ablative doses as described herein can be delivered with radiotherapy system as are known to one of ordinary skill in the art. The radiotherapy system can be programmed with software instructions to treat the tumor with ablative and sub-ablative doses. The software instructions may comprise treatment planning software so as to control the locations and timing of the ablative and sub-ablative doses. Alternatively, or in combination, the radiotherapy may comprise brachytherapy. For example, radioactive seeds may be placed near a tumor such as a prostate tumor, and sub-ablative treatments can be applied with a radiotherapy machine at locations away from the prostate. The presently disclosed methods and apparatus can be combined with prior methods, apparatus and compounds for treating cancer, such as immunomodulatory therapy, for example.

Without being bound by any particular theory, it is believed that ablative doses can release large amounts of immunogenic antigens from cancer cells, and subsequent treatment with the sub-ablative dose may cause the cancer cells exposed to the sub-ablative dose to present similar antigens, so as to trigger an immune response. Also, the treatment with the subsequent sub-ablative dose may play a role with respect to the tumor micro-environment and decrease the extent to which the tumor provides an immune-privileged site.

Differing schedules and doses of radiation on tumors may have differing immune-modulatory effects. Treatment plans of longer than seven days can be immunosuppressive and single ablative doses, releasing large amounts of antigen can be immunogenic. The current disclosure provides radiation schemes in the treatment of solid tumors, leading to increased immunogenicity and increased accessibility of the tumor to immune cells and therapeutic agents. In some embodiments the radiation schemes described herein combine non-ablative immune priming followed by ablation, termed “Immune Priming Ablation” (IPA), and can lead to a more potent in situ vaccine.

Prior therapies have generally been ineffective in the treatment of certain solid tumors due in part to the tumor microenvironment behaving as an immune privileged site and leading to chemo- and radio-resistance. There are multiple factors contributing to the immunosuppressive tumor microenvironment including tumor-promoting immune cells, the desmoplastic reaction and the disorganized tumor vascular. There can be a large population of immunosuppressive stromal cells present in tumors, such as, myeloid derived suppressor cells (MDSCs), cancer associated fibroblasts (CAFs) and tumor associated macrophages (TAMs). TAMs and CAFs can be key players in the creation of excessive extracellular matrix (ECM) by collaborating to induce a fibrotic reaction, similar to “wound healing” response after injury. Tumor associated macrophages constitute a large portion of the resident immune cells in pancreatic cancer, influencing the inhibition of infiltrating cytotoxic T cells and generally can be characterized as M1, anti-tumorigenic, and M2, pro-tumorigenic. Dendritic cells (DCs) are a very small population of immune cells, but those residing in the tumor are often tolerogenic and lead to the induction of immunosuppressive regulatory T cells (T_{regs}) and inhibition of cytotoxic T cells. Many tumor cells down regulate the expression of major histocompatibility complex (MHC) Class I, and components of antigen presentation machinery, which can essentially prevent MHC-peptide presentation, thereby escaping recognition by immune effector cells. The desmoplastic reaction creates a fibrotic meshwork, which further hinders accessibility of cytotoxic immune cells and separates tumor cells and blood vessels, while also decreasing permeability.

The disorganized and inefficient tumor vasculature can also play a role in perpetrating the immunosuppressive tumor microenvironment and is created in part by the unregulated growth of the tumor. As the tumor progresses, angiogenesis is often unable to match the rate of growth and therefor a structured vessel network is unable to effectively form. The tumor vasculature is also characterized by immature and leaky vessels and contributes to the increased interstitial pressure seen in many solid tumors, which can lead to decreased immune cell extravasation. This disorganization can help lead to chemo- and radio-resistance due to increased hypoxia and lack of accessibility of not only drugs, but immune infiltration as well.

Radiation therapy has a well-established role in local tumor control through direct cell death. Anecdotal evidence suggests that tumor ablation can on rare occasions produce a response at distant sites, this has been postulated to arise from the induction of systemic immunity. Radiation has been shown to induce immunogenic cell death (ICD) and can be the first step in anti-tumor immunity.

Damage and cell stress caused by radiation treatment can promote activation and maturation of dendritic cells (DCs). DCs are antigen-presenting cells (APCs) that engulf antigen from their environment and present it to T cells on MHC Class I or II receptors. Activation of T cells depends not only on the presentation of antigen from DCs but also on co-stimulatory molecules, and is one of the most important phases of adaptive-dependent anti-tumor immunity. Activation of T cells can often be ineffective because of the immunosuppressive tumor microenvironment. Harnessing radiation's activating potential while limiting its negative impact in accordance with embodiments described herein can lead to much more effective clinical outcomes.

There are three dosing regimens applicable clinically in the treatment of cancer: conventional fractionation, sub-ablative hypo-fractionation and ablative hypo-fractionation (Table 1). These prior dosing regimens can be combined with sub-ablative treatments in accordance with some embodiments. Conventional fractionation in the treatment of cancer consists of many low dose fractions delivered over longer periods of time (more than seven days) and is typically considered immunosuppressive, repeatedly killing any radiosensitive infiltrating immune cells. Sub-ablative hypo-fractionation consists of larger non-lethal doses delivered in under seven days and has some immune-modulatory effects. While this regimen increases the anti-tumor immune response, cytotoxicity of the treatment alone is not as effective at controlling tumor growth. Ablative hypo-fractionation causes direct cell death, releasing large amounts of antigen, and can control local tumor growth. The disadvantage of a single ablative fraction RT is the induction of a pro-tumor fibrotic response, orchestrated in part by tumor associated macrophages in a tumorigenic phenotype secreting TGFβ, and the attraction of immunosuppressive cells to the tumor microenvironment, which can diminish the anti-tumor response. The effects of different forms of fractionated radiotherapy are summarized in Tables 1 and 2 and suitable for combination in accordance with some embodiments.

Treating tumors with a combination of an ablative treatment (e.g. a single ablative treatment) and one or more sub-ablative treatments can combine the benefits of both the ablative treatment that generates antigens and kills cells with the benefits of the sub-ablative treatment that improves immune access to the tumor site and activates the immune system. The initial ablative treatment results in an increase in tumor antigen release, and in damage associated molecular pattern expression and an up-regulation of MHC Class I on surviving tumor cells, thus reversing immune escape of the tumor. Following this treatment with one or more sub-ablative doses over a short period of time, for example less than seven days, can provide further immune-modulatory benefits including normalization of vasculature, up-regulation of damage associated molecular patterns, MHC Class I, and adhesion markers and increased release of chemokines, thus attracting effector T cells to the tumor, and reducing the influx of immunosuppressive regulatory T cells. The effects of these two treatments may be synergistic, resulting in a much larger therapeutic effect than would be expected from the effect of either alone. Hypothesized effects of this treatment are summarized in Table 2.

TABLE 1
Clinically utilized radiation fractionation schemes

| | Clinical Treatment | Hypothesized Effect |
|---|---|---|
| Conventional | 1.8 Gy × 28 = 50.4 Gy | Immunosuppressive, poor |
| Fractionation | administered over | tumor and T cell response - |
| | more than two weeks | treatment time greater |
| | | than 7 days kills |
| | | infiltrating T cells |
| Sub-ablative | 8 Gy × 3 = 24 Gy | Immunomodulatory, but |
| hypo- | | inefficient tumor control |
| fractionation | | |
| Ablative hypo- | 24 Gy × 1 = 24 Gy | Induces secondary wound |
| Fractionation | 18-20 Gy × 3 = ~60 Gy | healing fibrotic response |
| | | and resistance |

TABLE 2
Experimental radiation schemes

| | | Hypothesized |
|---|---|---|
| Radiation Schemes | Treatment Scheme | Result |
| Immune Priming | 0.5 Gy × 4 + 22 Gy = 24 Gy | Increased |
| Ablation: | 1 Gy × 4 + 20 Gy = 24 Gy | immunogenicity, |
| Pre-priming | | plus in situ |
| | | vaccine |
| Immune Priming | 22 Gy + 0.5 Gy × 4 = 24 Gy | Increased |
| Ablation: | 20 Gy + 1 Gy × 4 = 24 Gy | immunogenicity, |
| Post-ablative | | plus in situ |
| modulation | | vaccine |

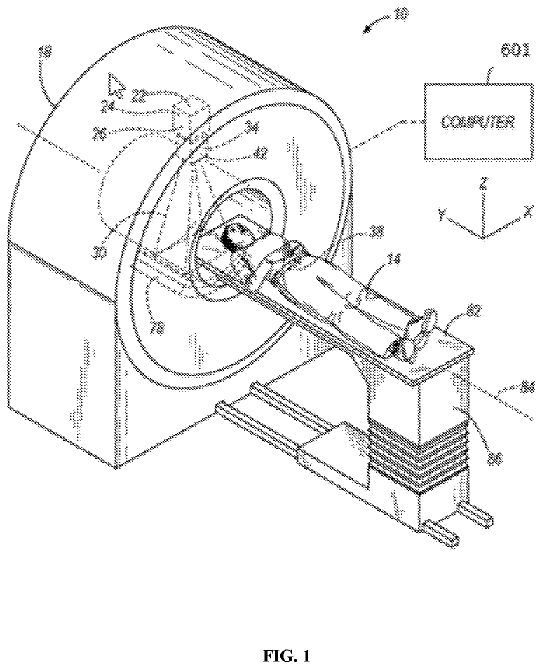

FIG. 1 illustrates a radiation therapy treatment system 10 that can provide radiation therapy to a patient 14 as described herein. The radiation treatment system 10 may comprise one or more components of many prior systems suitable for incorporation in accordance with the embodiments disclosed herein. Examples of prior systems suitable for incorporation in accordance with the present disclosure include systems from Accuray, such as the CyberKnife, the Radixact and TomoTherapy treatment systems, and systems from Varian such as the Edge Radiosurgery System, the TrueBeam Radiotherapy system, the Calypso Extracranial Tracking system, and Intracranial tracking. The therapy system may comprise tracking and imaging systems to align the patient with the tumor. The radiation therapy treatment can include photon-based radiation therapy, brachytherapy, electron beam therapy, proton, neutron, or particle therapy, or other types of treatment therapy. The treatment system 10 comprises a digital processing device 601 to control the beam energy levels and doses delivered to the subject, for example with stereotactic radiation therapy (STRT). The treatment system 10 may also comprises imaging components and systems as is known to one of ordinary skill in the art. The radiation therapy treatment system 10 includes a gantry 18 coupled to the computer to control the beam placement, although other beam directing devices can be used. The gantry 18 can support a radiation module 22, which can include a radiation source 24 and a linear accelerator 26 coupled to the computer and operable to generate a beam 30 of radiation. Though the gantry 18 shown in the drawings is a ring gantry, i.e., it extends through a full 360° arc to create a complete ring or circle, other types of mounting arrangements may also be employed. For example, a C-type, partial ring gantry, or robotic arm could be used. Any other framework capable of positioning the radiation module 22 at various rotational and/or axial positions relative to the patient 14 may also be employed. In addition, the radiation source 24 may travel in path that does not follow the shape of the gantry 18. For example, the radiation source 24 may travel in a non-circular path even though the illustrated gantry 18 is generally circular-shaped.

The radiation module 22 can also include a modulation device 34 operable to modify or modulate the radiation beam 30. The modulation device 34 provides the modulation of the radiation beam 30 and directs the radiation beam 30 toward the patient 14. Specifically, the radiation beam 34 is directed toward a portion of the patient. Broadly speaking, the portion may include the entire body, or may be smaller than the entire body and can be defined by a two-dimensional area and/or a three-dimensional volume. A portion desired to receive the radiation is an example of a region of interest. The region of interest, 38, may comprise a first treatment site, a second treatment site, or a subsequent treatment site. The region of interest 38 may also include a margin around or partially around the target. Another type of region of interest is a region at risk of radiation damage. If a portion includes a region at risk of radiation damage, the radiation beam is preferably diverted from the region. The patient 14 may have more than one region that receives radiation therapy as described herein.

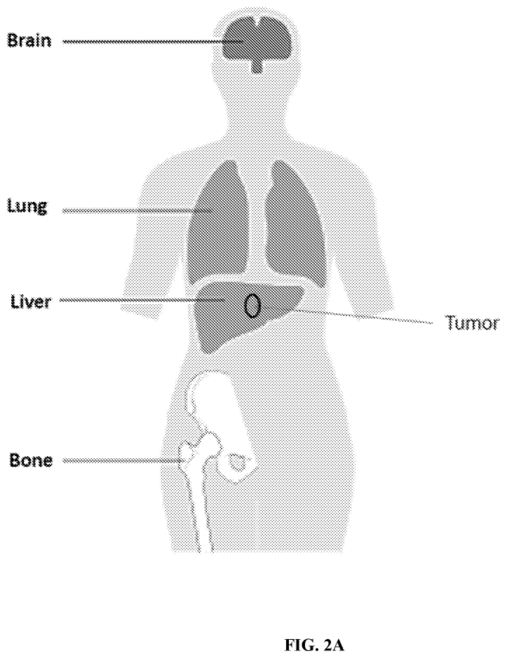

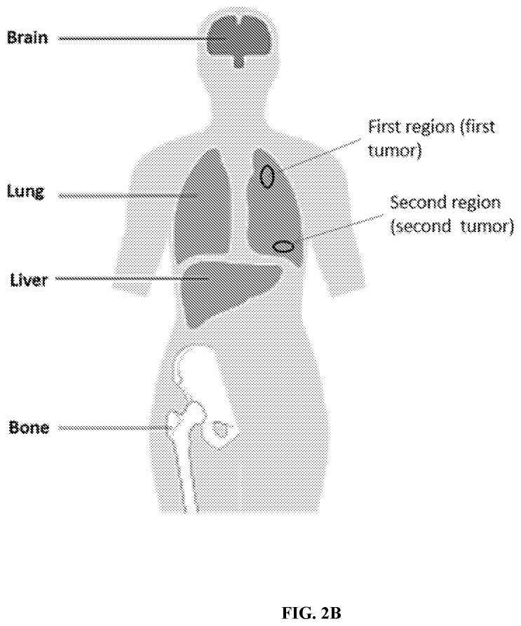

FIG. 2A illustrates an example of a patient with a tumor in a first region, in this case the liver. Using the methods described herein this tumor may be treated with an ablative dose of radiotherapy administered to the entire tumor or a portion thereof, followed by one or more sub-ablative doses of radiotherapy to the same tissue. In some cases, the methods of this disclosure may contemplate administering a first ablative dose of a radiotherapy to a whole, or a part of, a tumor, followed by administering one or more sub-ablative doses to the tumor or another location or locations of the subject. The sub-ablative doses may be administered to the whole tumor, or to a part of the tumor. If the ablative dose was administered to a part of the tumor then the sub-ablative doses may be administered to the same part of the tumor, or to a different part of the tumor.

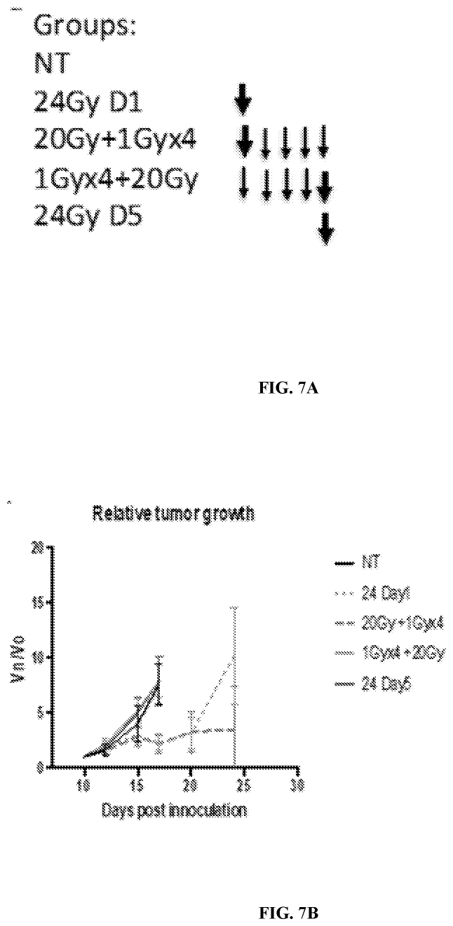

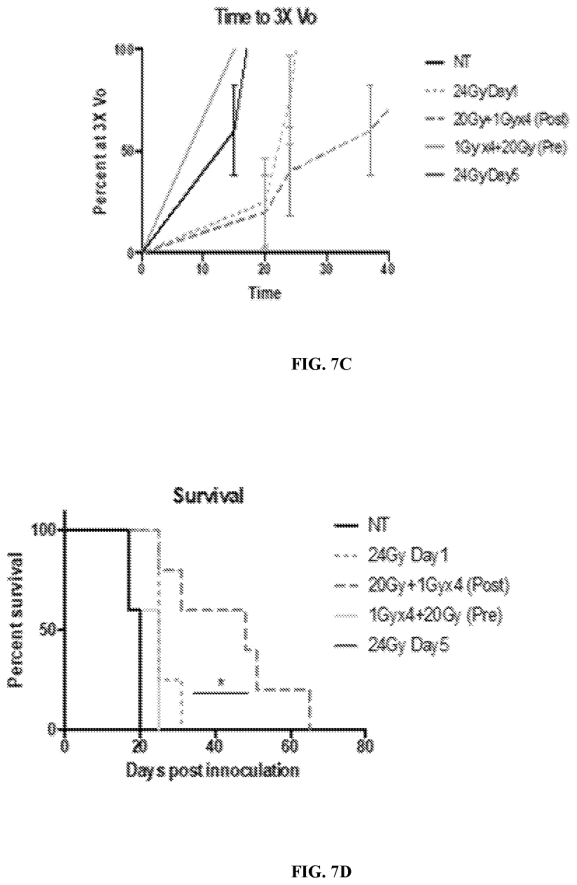

The sub-ablative doses of the radiotherapy, delivered subsequent to the ablative dose, can increase the immune reaction against the tumor and improve the treatment response. For example, as shown in FIG. 7A, mice with tumors were administered either no treatment, a single ablative dose of radiotherapy to the tumor, or a single ablative dose of radiotherapy followed up four sub-ablative doses of radiotherapy to the tumor. The mice which received the ablative dose followed by sub-ablative doses had an improved survival rate. Mice which received the ablative and sub-ablative doses in the reversed order, four sub-ablative doses followed by a single ablative dose, had a slightly better survival rate than mice treated with a single ablative dose alone, but it was not as great as when the ablative dose was performed before the sub-ablative doses. Similar approaches can be used for the treatment of patients such as human and animal patients, for example with appropriate software instructions on a radiotherapy system as described herein.

FIG. 2B illustrates an example of a patient with two tumors in an organ such as the lung. The two tumors may comprise a first tumor and a second tumor. The first tumor may comprise a primary tumor, and the second tumor may comprise a metastatic or recurrent tumor. Alternatively, the first tumor may comprise a metastatic or recurrent tumor and the second tumor may comprise the primary tumor. Using the methods described herein the first tumor may be treated with an ablative dose of radiotherapy administered to the entire tumor or a portion thereof. Subsequently, the first and second tumors may each be treated with one or more sub-ablative doses of radiotherapy. The subsequent, sub-ablative doses of the radiotherapy can increase the immune reaction against the tumors and improve the treatment response as described herein. In this case, the second tumor may stop growing or regress despite not having been administered an ablative dose. Using the methods described herein, a patient may have a first tumor, and any number of secondary tumors. A first ablative dose of radiotherapy may be administered to the first tumor, followed by one or more sub-ablative doses of radiotherapy to some or all of the secondary tumors. Sub-ablative doses of radiotherapy may also be administered to the first tumor after the administration of the ablative dose of radiotherapy. The secondary tumors may be in the same organ as the tumor treated with the ablative dose, or may be in one or more different organs. In some cases, the first tumor is selected based on its size, location, disease stage, or other clinically relevant properties.

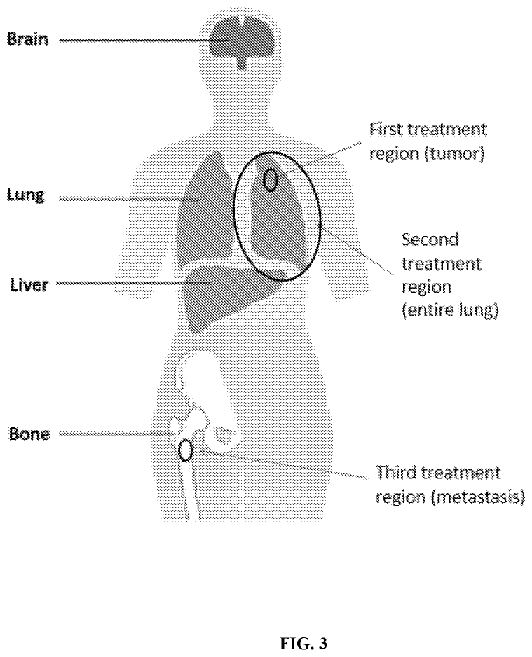

FIG. 3 illustrates an example patient with a tumor in the lung and a metastatic tumor in the bone. In some cases, the tumor in the lung may be treated with a single ablative dose of radiotherapy. Subsequent to the first dose the entire lung may be treated with one or more sub-ablative doses of radiotherapy. While treating the entire lung with the one or more sub-ablative doses, the bone with the metastatic tumor may also be treated with one or more sub-ablative doses. In another example, the location of the bone metastasis may be treated first with a single ablative dose of radiotherapy. Subsequent to this first dose the bone metastasis and either the tumor in the lung, or the entire lung, may be treated with one or more sub-ablative doses.

In some cases, after treatment of a first tumor with an ablative dose, one or more sub-ablative doses may be administered to the entire body. In some cases, the one or more sub-ablative doses may be administered to an entire peritoneal cavity, for example. In some cases, the one or more sub-ablative doses may be administered to an entire organ which is known to contain one or more metastatic tumors, or to an entire organ which is known to be a likely site for the development of metastatic tumors. For example, patients with breast cancer develop metastatic tumors in the bones, lungs, liver or brain. Patients with lung cancer can develop metastatic tumors in the other lung, or in the adrenal gland, bones, brain and liver. Examples of organs which may be likely to develop metastatic tumors include, but are not limited to, bones, lymph node, lung, liver, brain, adrenal gland, breast, eye, kidney, muscles, pancreas, salivary gland, and spleen.

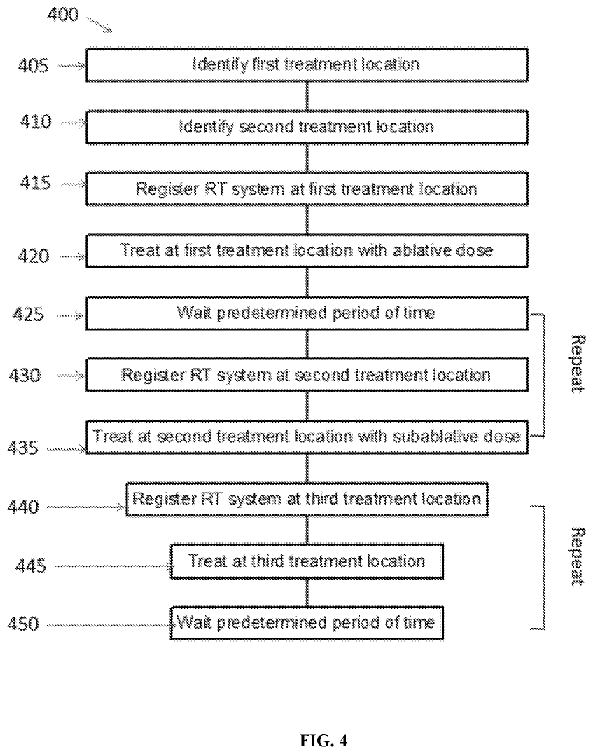

FIG. 4 illustrates an example process, 400, which may be used to treat a cancer. The first step of the process, 405, comprises identifying a first treatment region. The first treatment region may comprise a region of a tumor within the patient. In some cases, the first treatment region can comprise a primary tumor. In some cases, the first treatment region can be the location of a metastatic tumor. In some cases, if several different tumors are present in the patient the first treatment region may comprise the location of the tumor which is most accessible for radiation treatment. In some cases, if several different tumors are present in the patient the first treatment region may be the location of the tumor which is safest to treat with radiation. The tumor which is safest to treat with radiation may be the tumor which is located such that adverse reactions from the radiation are likely to be decreased as compared with other locations. In some cases, the first treatment region comprises the location of the largest tumor in the patient. In some cases, the first treatment region comprises the location of the only known tumor in the patient.

After the first region has been identified, a second treatment region is identified at step 410. In some embodiments, the second treatment region may comprise a location which has a tumor. In some cases, the second treatment region may be a different region from the first treatment region. In some cases, the second treatment region may comprise the same region as the first treatment region. In some cases, the second treatment region may be within the same organ as the first treatment region, or in a different organ from the first treatment region. The second treatment region may be a location which has a secondary tumor, or a primary tumor. In some cases, the second treatment region has a smaller tumor than the first treatment region. In some cases, the second treatment region is a location which is difficult to treat with radiation. In some cases, the second treatment region is a location which is more sensitive to adverse effects of radiation treatment than the first treatment region. In some cases, the second treatment region is a location which does not have an identified tumor, but is known to be a likely area for the development of metastatic tumors.

The next step 415 comprises registering a radiotherapy system with a patient to target the first treatment region. The radiotherapy system may comprise components any radiotherapy system known in the art, such as the known radiotherapy systems commercially available from Varian Medical and Accuray, for example. For example, the radiotherapy system may comprise the system of FIG. 1, or a similar system. The radiotherapy type may comprise any type suitable for clinical use as described herein, for example x-ray radiation, gamma ray radiation, alpha particle radiation, beta particle radiation, neutron particle radiation, external beam radiation or brachytherapy. The radiotherapy system may include an imaging system which is able to image a tumor within the patient and target the tumor, for example.

Once the first treatment region is registered, it may be treated with an ablative dose of radiation at a step 420. An ablative dose may be a dose of between about 10 Gy and about 60 Gy, between about 20 Gy and about 40 Gy, or between about 20 Gy and about 30 Gy. In some cases, an ablative dose is about 10 Gy, 12 Gy, 14 Gy, 16 Gy, 18 Gy, 20 Gy, 22 Gy, 24 Gy, 26 Gy, 28 Gy, 30 Gy, 32 Gy, 34 Gy, 36 Gy, 38 Gy, 40 Gy, 42 Gy, 44 Gy, 46 Gy, 48 Gy, 50 Gy, 52 Gy, 54 Gy, 56 Gy, 58 Gy, or 60 Gy, or within a range defined by any two of the preceding values.

Following the first treatment, a step 425 in the process 400 involves waiting for a predetermined period of time before proceeding with a subsequent treatment. The predetermined period of time may be between about 1 hour and about 4 days, for example. In some cases, the predetermined period of time may be between about 1 hour and about 36 hours, between about 6 hours and about 30 hours, between about 12 hours and about 26 hours, between about 20 hours and about 28 hours, or between about 22 hours and about 26 hours. A person of ordinary skill in the art can determine an appropriate amount of time to wait in accordance with the presently disclosed teachings.

At a step 430, the radiotherapy system is registered at the second treatment region, and the second treatment region may be treated with a sub-ablative dose at a step 435. A sub-ablative dose may comprise a dose of between about 0.1 Gy and about 3 Gy, between about 0.2 Gy and about 2 Gy, between about 0.3 Gy and about 1 Gy, between about 0.3 Gy and about 0.7 Gy, between about 0.1 Gy and 0.5 Gy, or between about 0.8 Gy and about 1.2 Gy. Steps 425 to 435 may be repeated a number of times. In some cases, steps 425-435 are repeated two times, three times, four times, five times, six times or more than six times in order to induce an immunogenic response without ablation at the second treatment region or in additional sub-ablative treatment regions. In some cases, a sub-ablative dose comprises a plurality of sub-ablative doses and each of the plurality of sub-ablative doses comprises a dose of between about 0.1 Gy and about 3 Gy, between about 0.2 Gy and about 2 Gy, between about 0.3 Gy and about 1 Gy, between about 0.3 Gy and about 0.7 Gy, between about 0.1 Gy and 0.5 Gy, or between about 0.8 Gy and about 1.2 Gy. The radiotherapy system may next be registered at the third treatment region at a step 440, and the third treatment region treated with a sub-ablative dose at a step 445. The sub-ablative dose may be a dose of between about 0.1 Gy and about 3 Gy, between about 0.2 Gy and about 2 Gy, between about 0.3 Gy and about 1 Gy, between about 0.3 Gy and about 0.7 Gy, or between about 0.8 Gy and about 1.2 Gy. Steps 440 and 445 may be repeated any number of times if beneficial to inducing an immunogenic response without ablation at the third treatment region.

After treating at the third treatment region the system waits for a predetermined period of time, 450, and then may repeat steps 440-450 one, two, three, four, or more than four times. The predetermined period of time may be between about 1 hour and about 4 days. In some cases, the predetermined period of time may be between about 1 hour and about 36 hours, between about 6 hours and about 30 hours, between about 12 hours and about 26 hours, between about 20 hours and about 28 hours, or between about 22 hours and about 26 hours.

A processor as described herein can be configured with instructions to perform the method 400.

While FIG. 4 illustrates method 400 of treating a cancer in accordance with an embodiment, a person of ordinary skill in the art will recognize many adaptations and variations. One or more of the steps may be omitted, repeated, performed concurrently, and/or performed in a different order. In some embodiments, one or more of the steps may be modified, or may comprise sub-steps. In addition, a person of ordinary skill in the art will appreciate that additional steps may be included in performing this method.

In some cases, a cumulative amount of radio therapy delivered to the second region throughout a course of treatment comprises less than an ablative dose. In some cases, the cumulative sub-ablative amount comprises a plurality of sub-ablative doses.

FIG. 5A illustrates biological processes which may result from pre-priming a tumor with one or more sub-ablative doses of radiotherapy before administering an ablative dose of radiotherapy, in accordance with some embodiments. In this case a tumor, shown with blood vessels and immune cells (dark circles), is treated first with four sub-ablative doses (such as 0.5 Gy or 1 Gy, depicted by small ‘lightning bolts’), and then treated with a single ablative dose (such as 22 Gy or 20 Gy, depicted by the large ‘lightning bolt’). The pre-priming sub-ablative doses may cause sensitization of the tumor to immune cells. This may be evidenced by increased expression of stress markers, immunomodulation/immunogenic cell death and differences in peptide reservoir and MHC class I expression. The tumor is next treated with an ablative dose of radiotherapy, this treatment results in cell death, particularly of tumor cells and releases tumor antigens into the tumor microenvironment and circulation. The ablative radiotherapy will also result in other changes to the surviving cells of the tumor, such as increased expression of damage associated molecular patterns, and increased cytokine release, which are pro-immunogenic. The release of tumor antigens stimulates an effector T cell response, which is facilitated by the prior sensitization of the tumor achieved by the sub-ablative doses.

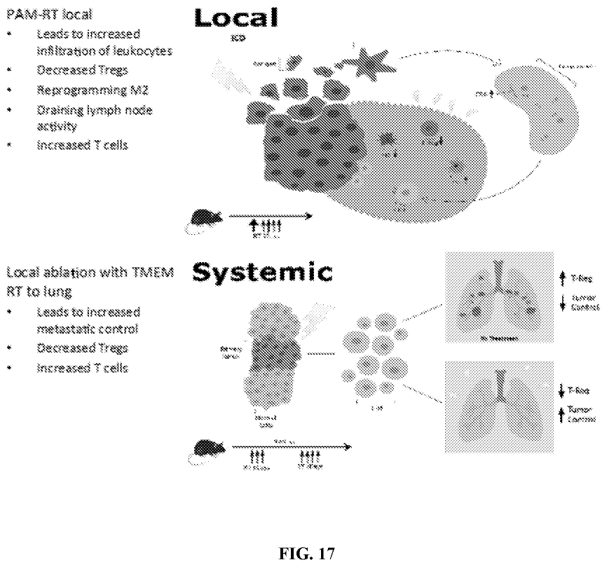

FIG. 5B illustrates an example of post-ablative modulation and the biological processes involved. In this case a tumor, shown with blood vessels and immune cells (dark circles), is treated first with a single ablative dose (such as 22 Gy or 20 G, depicted by the large ‘lightning bolt’), and then treated with four sub-ablative doses (such as 0.5 Gy or 1 Gy, depicted by small ‘lightning bolts’). The initial, ablative, dose of radiotherapy causes cell death and release of antigens which stimulate an effector T cell response. The subsequent, sub-ablative, doses of radiotherapy modulate the tumor environment and increase infiltration of the activated effector T cells into the tumor. The increased infiltration is facilitated by changes to the tumor caused by the sub-ablative treatments, including, normalizing vasculature (see FIG. 7B), altered cytokine milieu, and macrophage repolarization/reprogramming. As shown in FIG. 7A both pre-priming treatments and post-ablative modulation increased survival of tumor bearing mice. However post-ablative modulation showed a much greater impact on survival. FIG. 17 provides a summary of the effect of PAM-RT treatment on local and systemic immunomodulation.

The methods and systems disclosed herein may be used to treat a patient. In some cases, the patient is a human. In some cases, the patient has been diagnosed with a cancer. In some cases, the patient has been diagnosed with a solid cancer. Examples of types of cancer include, but are not limited to: esophageal cancer, breast cancer, gastric cancer, cholangiocarcinoma, pancreatic cancer, colon cancer, lung cancer, thymic carcinoma, mesothelioma, ovarian cancer, and endometrial cancer. In some cases, the first region may comprise a region of a primary tumor of an organ. Examples of organs which may develop a cancer include, but are not limited to, breast, bladder, brain, colon, rectal, endometrial, kidney, pancreas, prostate, liver, lung, skin, thyroid, uterus, lymph node, tonsil, thymus, spleen and bone marrow. In some cases, the patient may have more than one different types of cancer. In some cases, the patient has at least one detected tumor. In some cases, the patient's cancer has not responded to another therapy, or has stopped responding to another therapy.

In some embodiments, described herein is a method of treating a cancer in a subject, the method comprising providing an ablative dose of radiation therapy to a first region comprising the cancer followed by a sub-ablative dose to a second region, wherein the sub-ablative dose is administered after the ablative dose. In certain embodiments, the sub-ablative dose is administered at least 1 hour after the ablative dose. In certain embodiments, the sub-ablative dose is administered at least 1 day after the ablative dose. In certain embodiments, the sub-ablative dose is administered no more than 4 days after the ablative dose.

### Digital Processing Device

In some embodiments, the platforms, systems, media, and methods described herein include a digital processing device, or use of the same. In further embodiments, the digital processing device includes one or more hardware central processing units (CPUs), general purpose graphics processing units (GPGPUs), or field programmable gate arrays (FPGAs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

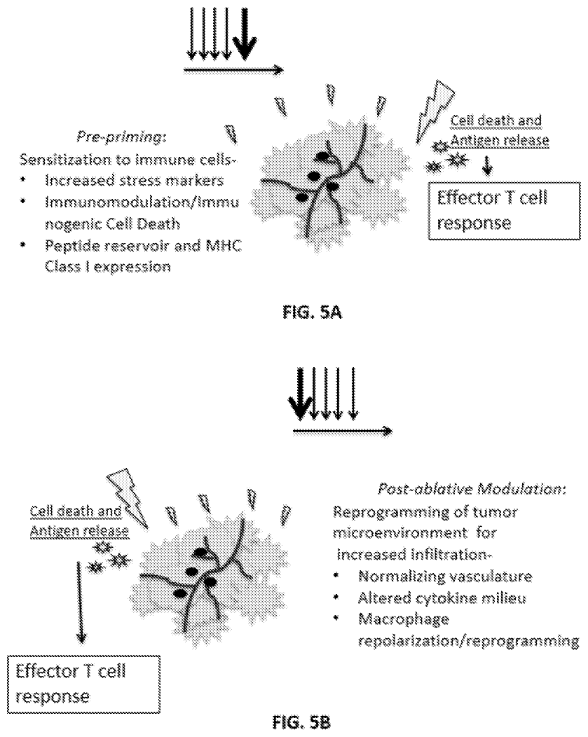

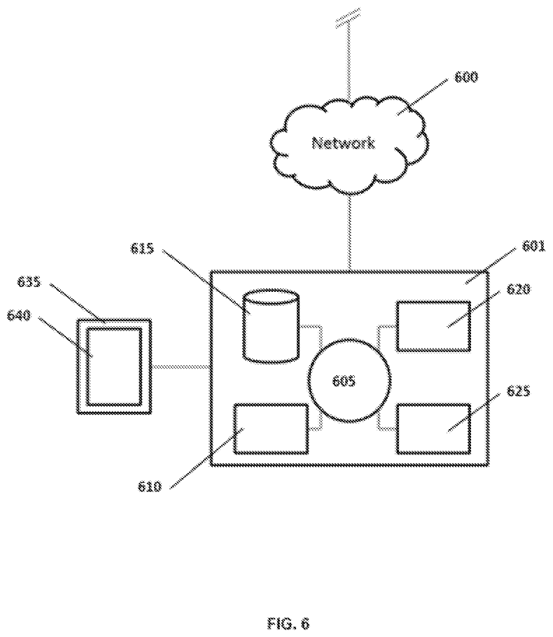

Referring to FIG. 6, in a particular embodiment, an exemplary digital processing device 601 is programmed or otherwise configured to a radiotherapy device as described herein. The device 601 can regulate various aspects of the radiotherapy device of the present disclosure, such as, for example, performing processing steps. In this embodiment, the digital processing device 601 includes a central processing unit (CPU, also “processor” and “computer processor” herein) 605, which can be a single core or multi core processor, or a plurality of processors for parallel processing. The digital processing device 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The digital processing device 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the device 601, can implement a peer-to-peer network, which may enable devices coupled to the device 601 to behave as a client or a server.

Continuing to refer to FIG. 6, the CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and write back. The CPU 605 can be part of a circuit, such as an integrated circuit. One or more other components of the device 601 can be included in the circuit. In some cases, the circuit is an application specific integrated circuit (ASIC) or a field programmable gate array (FPGA).

Continuing to refer to FIG. 6, the storage unit 615 can store files, such as drivers, libraries and saved programs. The storage unit 615 can store user data, e.g., user preferences and user programs. The digital processing device 601 in some cases can include one or more additional data storage units that are external, such as located on a remote server that is in communication through an intranet or the Internet.

Continuing to refer to FIG. 6, the digital processing device 601 can communicate with one or more remote computer systems through the network 630. For instance, the device 601 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

In some embodiments, disclosed herein is a computer readable medium configured with instructions that, when executed, cause a processor to provide instructions to a radiotherapy system to deliver an ablative dose of radiation therapy to a first region followed by delivery of a sub-ablative dose to a second region after the ablative dose. In some embodiments, the sub-ablative dose is delivered at least 1 hour after the ablative dose. In some embodiments, the sub-ablative dose is delivered at least 1 day after the ablative dose. In some embodiments, the sub-ablative dose is delivered no more than 4 days after the ablative dose.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™ and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash HTML 5, Apple® QuickTime®, Microsoft® Silverlight Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser. Software modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of information. In various embodiments, suitable databases include, by way of non-limiting examples, relational databases, non-relational databases, object-oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Example 1 Local PAM-RT Delays Tumor Growth and Increases Survival in Mice

To investigate the efficacy of PAM-RT for local tumor control, C57BL/6 mice with palpable, subcutaneous, 3LL tumors were divided into five treatment cohorts for pilot studies—untreated, 24 Gy at day 1 or day 5 of treatment, 1 Gy×4 followed by 20 Gy or 20 Gy followed by 1 Gy×4. Basically, four fractions of 1 Gy delivered either before or after a single fraction of 20 Gy to the primary tumor were compared with a 24 Gy single fraction of RT. When compared to single-dose RT, pre-treatment with low-dose RT showed minimal tumor control, but there was a significant growth delay and improvement in survival after PAM-RT of 1 Gy×4 fractions, as shown in FIGS. 7A-D.

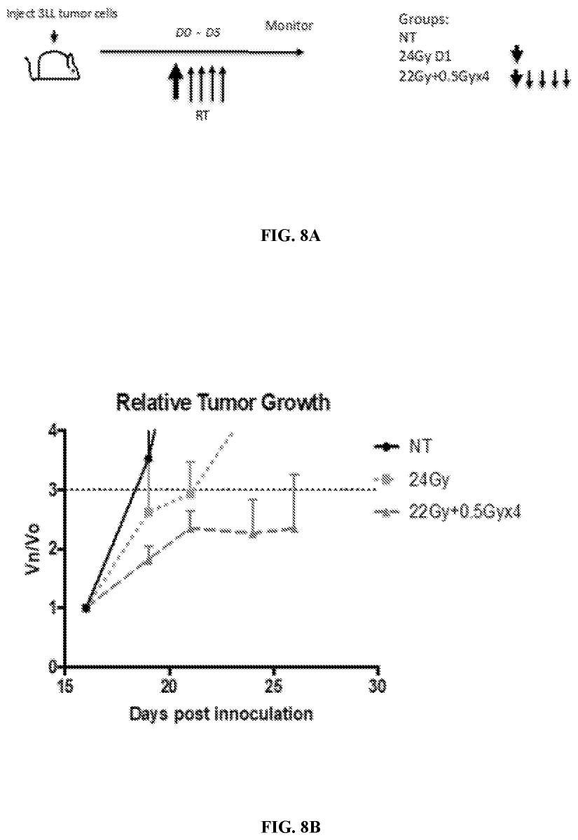

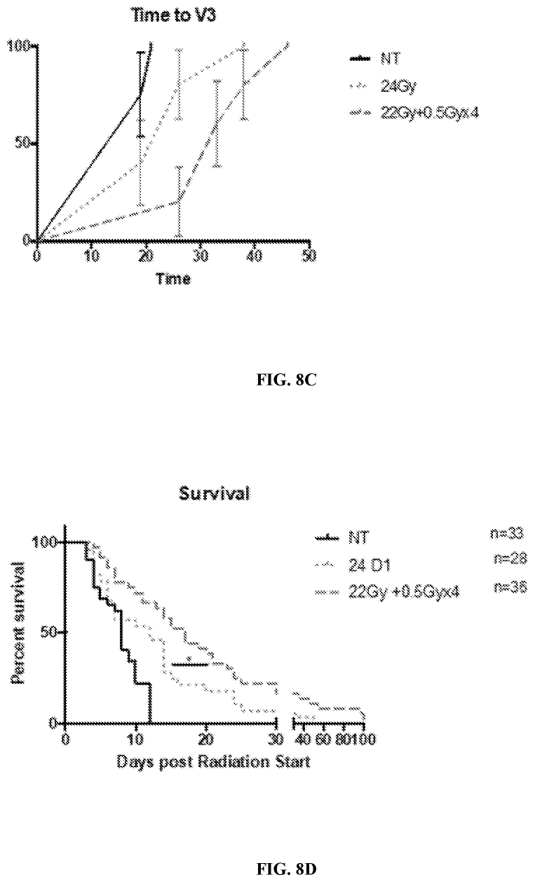

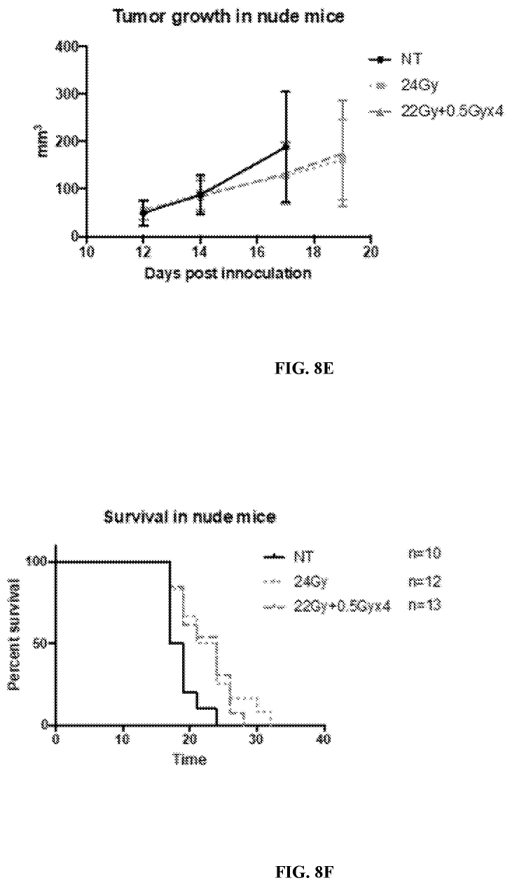

Since 3LL tumors grow rapidly, priming-RT efficacy could have been impacted by the tumor size difference for ablative RT on the fifth day of treatment. Further studies of tumor growth led to the adoption of PAM-RT with 0.5 Gy×4 fraction as the optimal modulation doses for all further experiments. As before, a single-dose ablative fractionation of 24 Gy was compared with 22 Gy followed by 4 fractions of 0.5 Gy PAM-RT. A schematic of the dosing schedule is shown in FIG. 8A. When compared to 24 Gy, PAM-RT delayed tumor growth as seen in the growth curve in FIG. 8B and the time to triple volume shown in FIG. 8C. Further, PAM-RT significantly improved survival in these animals, as seen in FIG. 8D. Although, a single fraction of 24 Gy is unable to cure 3LL tumor growth, significant growth delay was seen in mice bearing large 3LL tumors after PAM-RT, where single fraction ablative RT had little effect. Finally, the effects of PAM-RT were lost in immune compromised (nude) mice. FIG. 8E shows tumor growth in 3LL tumor bearing nude mice, and FIG. 8F shows survival in these mice. These data demonstrated the ability of PAM to delay local tumor progression and significantly increase survival in 3LL tumor bearing mice, although all mice eventually succumbed to local tumor growth. Furthermore, the PAM-RT effects were contingent on a competent immune system.

### Immunomodulatory Effects of Local PAM-RT In Vitro

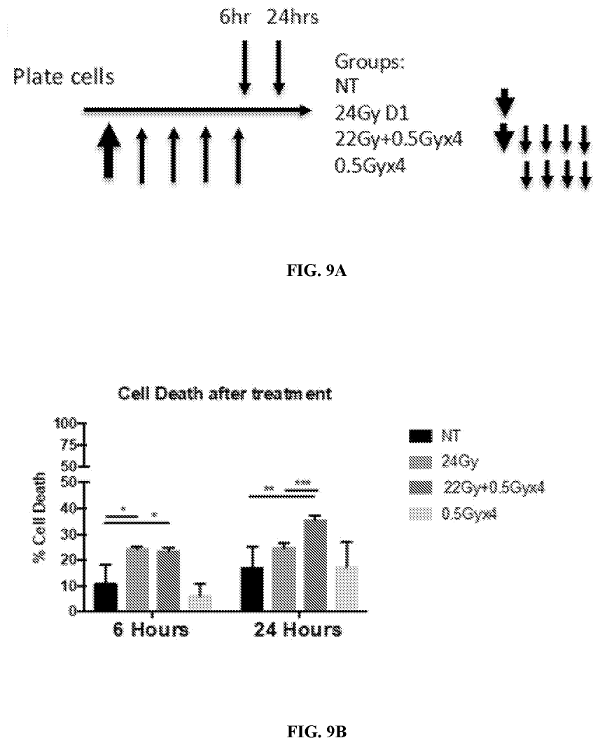

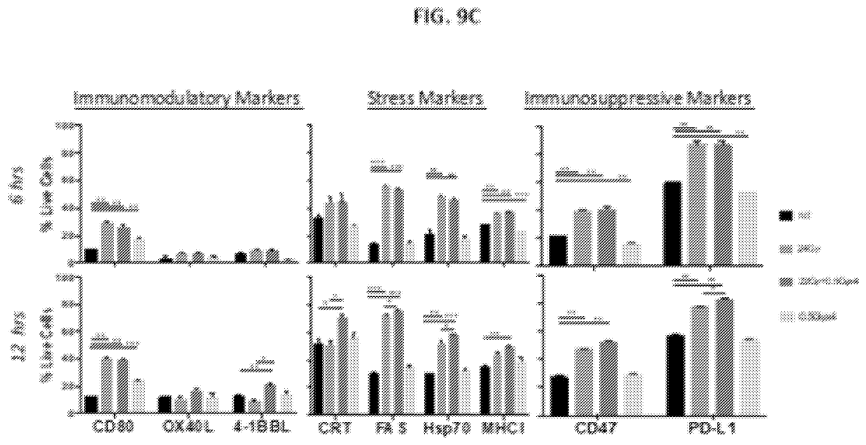

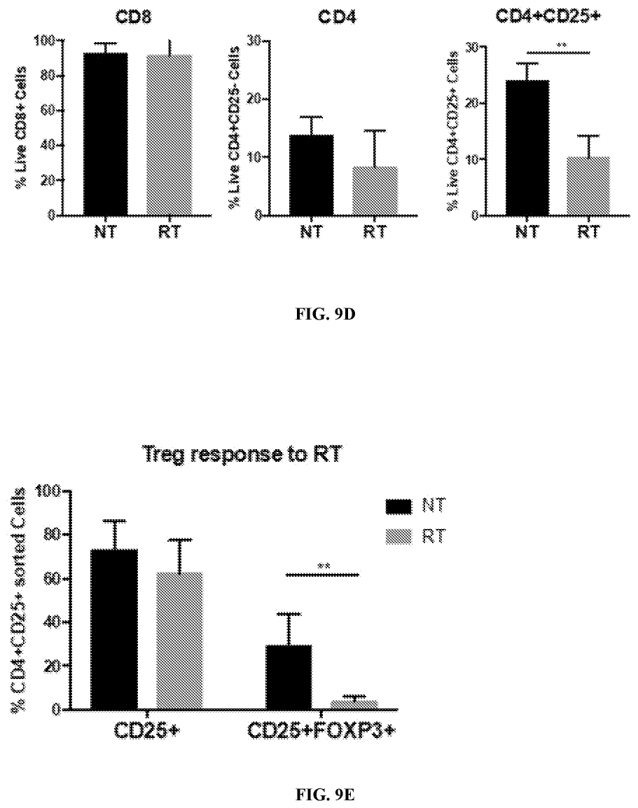

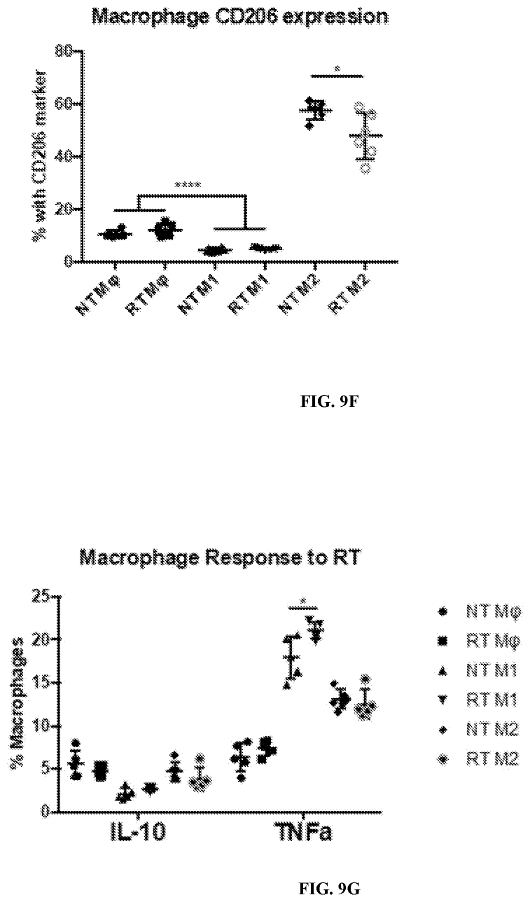

The immunomodulatory effects of PAM-RT on 3LL cells were also studied in vitro. An experimental scheme is shown in FIG. 9A. While no difference in cell death seen 6 hours after treatment, PAM-RT caused significantly more cell death at 24 hours than ablation alone (FIG. 9B). When compared to untreated cells and low-dose radiation-treated cells, ablative radiation, with or without PAM-RT, increased cell surface immunomodulatory markers (CD80), stress markers (CRT, Hsp70, Fas and MHC I) and immuno-suppressive markers (CD47 and PDL1) at 6-hour post-RT. Interestingly at 24 hours, PAM-RT treated cells had greater cell surface expression of 4-1BBL, CRT, Fas, Hsp70, and PD-L1, as compared to ablation alone, as seen in FIG. 9C.

To examine the effect of PAM-RT on immune cells in vitro bone marrow-derived macrophages and splenic T cells were harvested from wild-type C57Bl/6 mice. Macrophages were polarized with cytokines to M1 or M2 differentiation, or left untreated. Splenic T cells were sorted into the three main T cell populations: CD8+, CD4+CD25−, and CD4+CD25+ (Tregs). All populations were treated with low dose RT (0.5 Gy×4), one day after polarization or sorting. Ablative doses were not included in treatment due to the radio-sensitivity of immune cells. Characterization of T cell populations 6 hours after the final dose showed a significant decrease in Treg viability, which was not seen in the CD8 or CD4+CD25− T cells populations, see FIG. 9D. Within the sorted Treg population, cells treated with low-dose RT showed a trend towards decreased CD25 surface expression and significantly decreased FOXP3 and CD25 dual expression, shown in FIG. 9E. As shown in FIG. 9F, M2 polarized macrophages treated with low-dose RT showed a significant decrease in CD206 expression, a M2 marker. M2 polarized macrophages also tended to express less IL-10 after RT, indicating a repolarization after treatment, see FIG. 9G. These data indicate PAM-RT increases the cytotoxicity of ablative RT without inhibiting the immunomodulatory effects. Furthermore, our low-dose RT reduces Treg and reprograms M2 macrophages towards a less immunosuppressive phenotype.

### Local PAM-RT Promotes Tumor Microenvironment Remodeling by Reducing Treg and M2 Macrophages

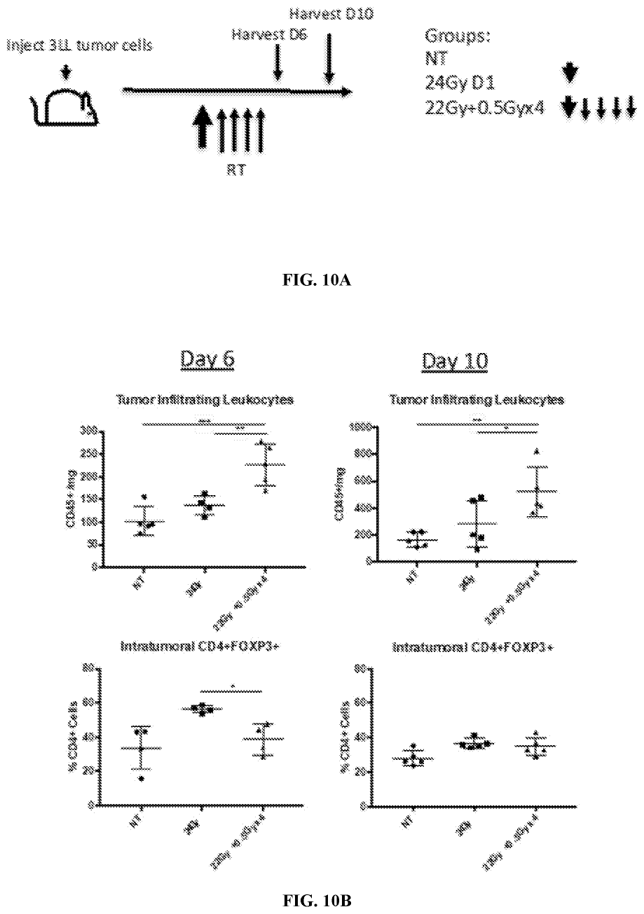

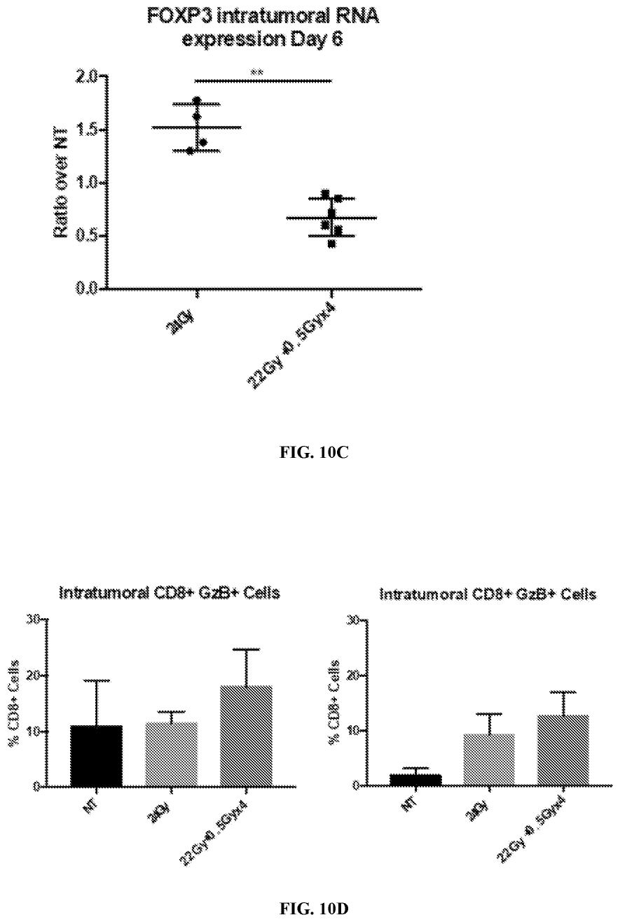

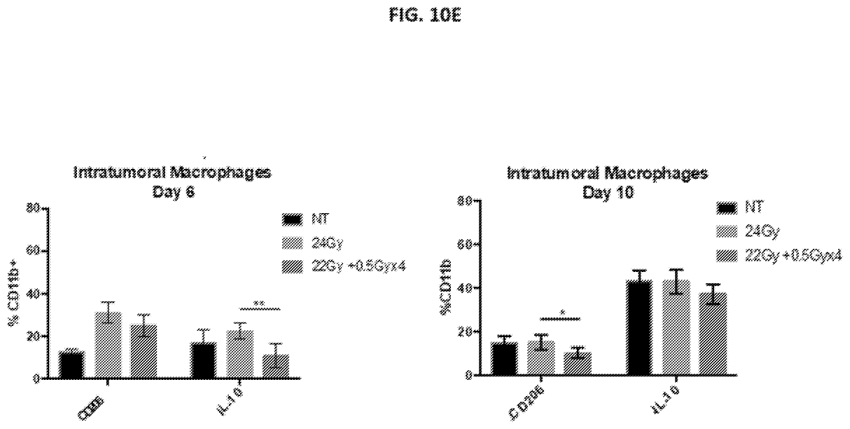

To investigate the immunological consequences of PAM-RT on the tumor microenvironment (TME), irradiated 3LL tumors were harvested on day 6 and day 10 after start of RT, as shown in FIG. 10A. There was a significant increase in leukocyte infiltration in tumors treated with PAM-RT, as compared to ablative RT alone. Upon phenotyping of the infiltrating leukocytes, there was a significant decrease in intra-tumoral Tregs, see FIG. 10B, and significantly increased CD8/Treg ratio at day 6 in PAM-RT treated mice, as compared to ablative RT alone. RT-PCR of whole tumor RNA showed a significant decrease in FOXP3 mRNA expression in PAM-RT treated tumors at day 6, as seen in FIG. 10C. As seen in FIG. 10D this remodeling was accompanied by a trend towards an increase in granzyme B secreting intratumoral CD8+ T cells at day 6 and day 10, indicating an increase towards effector CTL responses. Characterization of the intratumoral myeloid cell population revealed a significant decrease in IL-10 secreting macrophages and a slight decrease in CD206 expression at day 6 as well as a trend towards a decrease in IL-10 secretion and a significant decrease in CD206 expression at day 10 after start of treatment, as shown in FIG. 10E. Taken together, PAM-RT promotes TME remodeling by reducing immunosuppressive Treg and M2 macrophages, while increasing CTL activity.

### Local PAM-RT Increases Systemic T Responses and Decreases Tregs

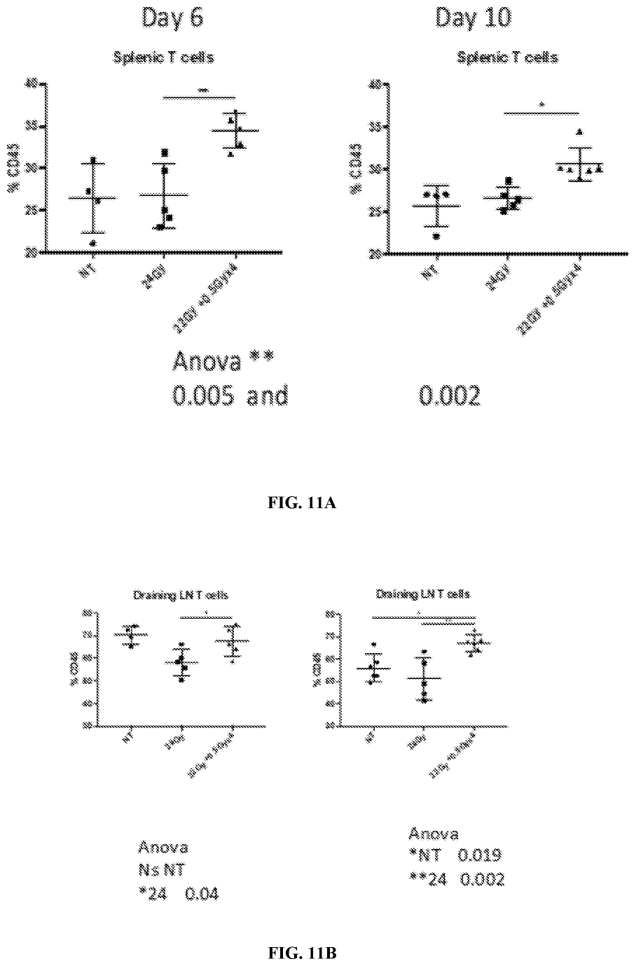

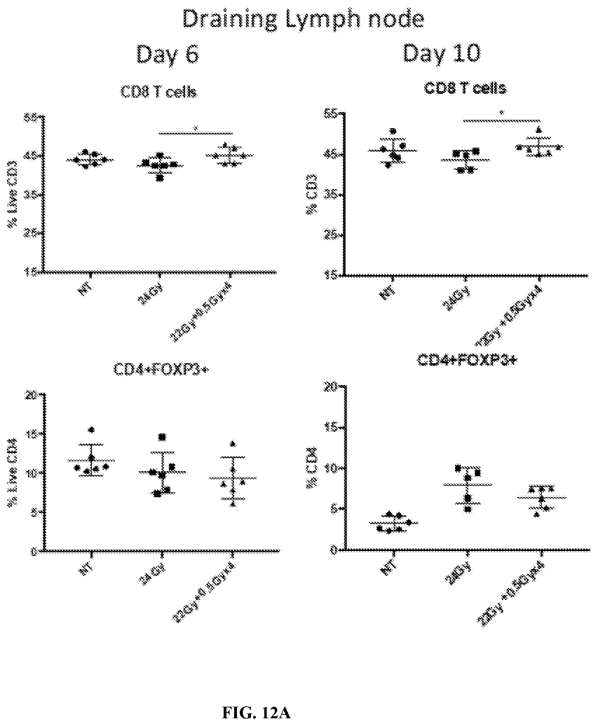

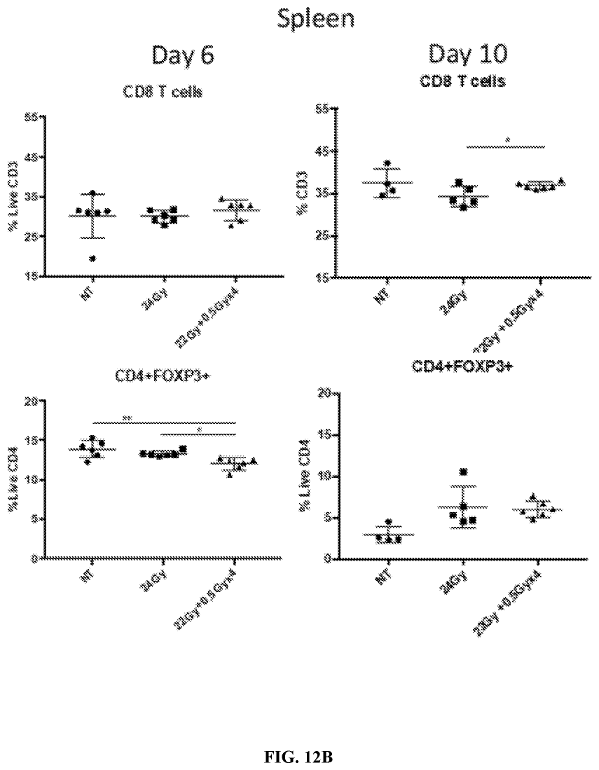

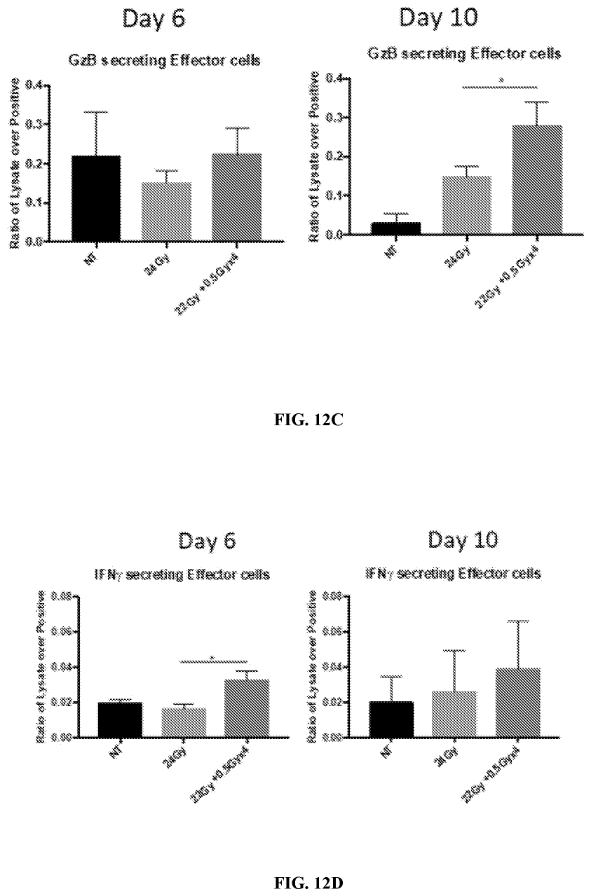

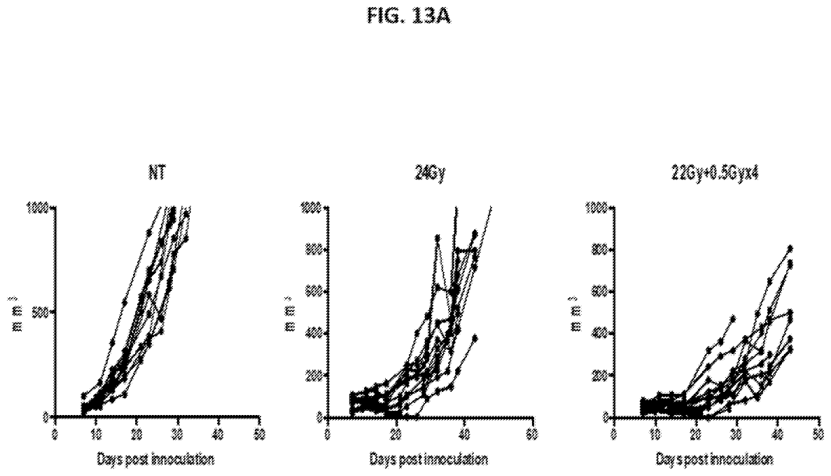

To examine the immune cells in the secondary lymphoid organs, tumor draining lymph nodes and spleens were harvested, 6 and 10 days after start of RT, as with previous studies (e.g. FIG. 10A). At 6 and 10 days, there was a significant increase in leucocytes in spleen and draining lymph nodes in PAM-RT treated mice, as seen in FIGS. 11A and 11B. In the tumor draining lymph node (TDLN) there were significantly more CD8+ cells and a trend towards a decrease of Tregs in PAM-RT treated mice at both days 6 and 10, as seen in FIG. 12A. Splenic analysis revealed minimal changes in CD8 T cells, but significant decreases in Tregs at day 6 which were reversed by day 10 with significantly more CD8+ T cells and minimal changes in Tregs in PAM-RT-treated mice, compared to a single ablative dose FIG. 12B. Investigations in the functional status of splenic T cells at day 6 and 10 with ELISPOTs revealed a trend toward increased granzyme B secreting effector cells, FIG. 12C, and a significant increase in IFNγ secreting effector cells, FIG. 12D, at day 6 in PAM-RT treated mice. Day 10 analysis revealed a significant increase in granzyme B secreting effector cells, and a trend toward increased IFNγ secreting effector cells. Although treatment in these mice was localized to the primary tumor, systemic modulation occurred similar to trends occurring in the tumor with decreased immunosuppression and increased T cell responses.

### Systemic PAM-RT Delays Metastatic Progression and Increases Survival

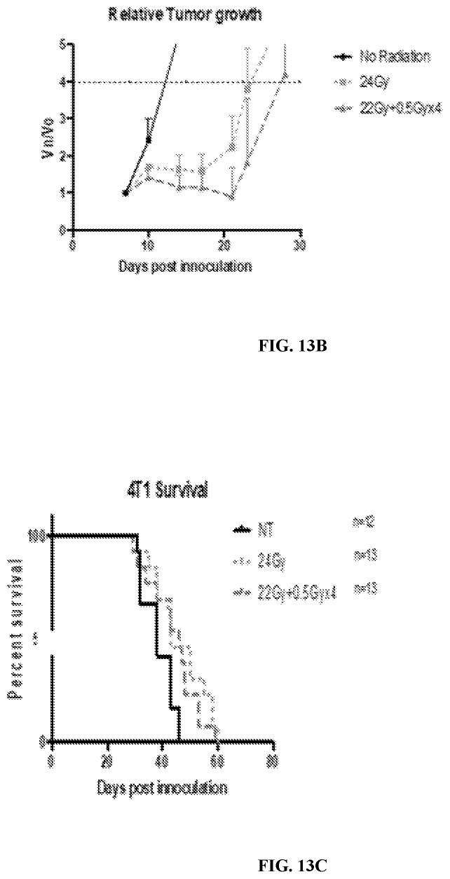

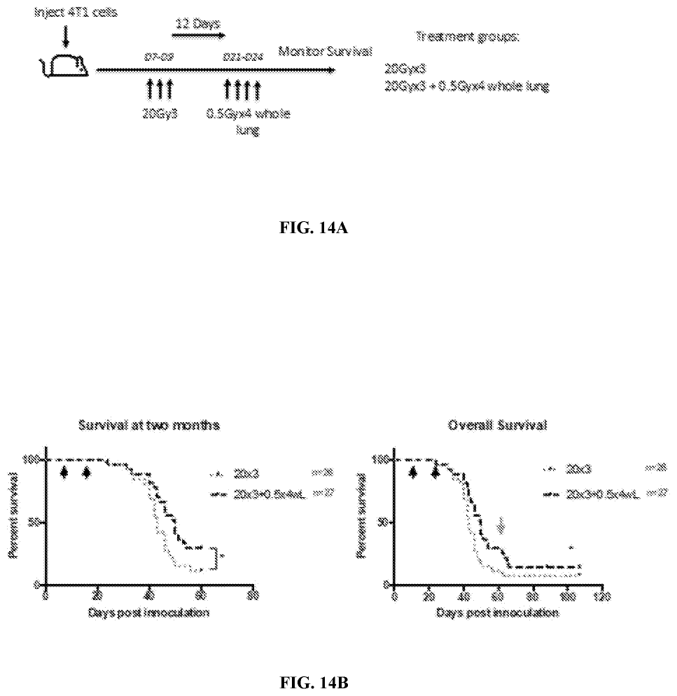

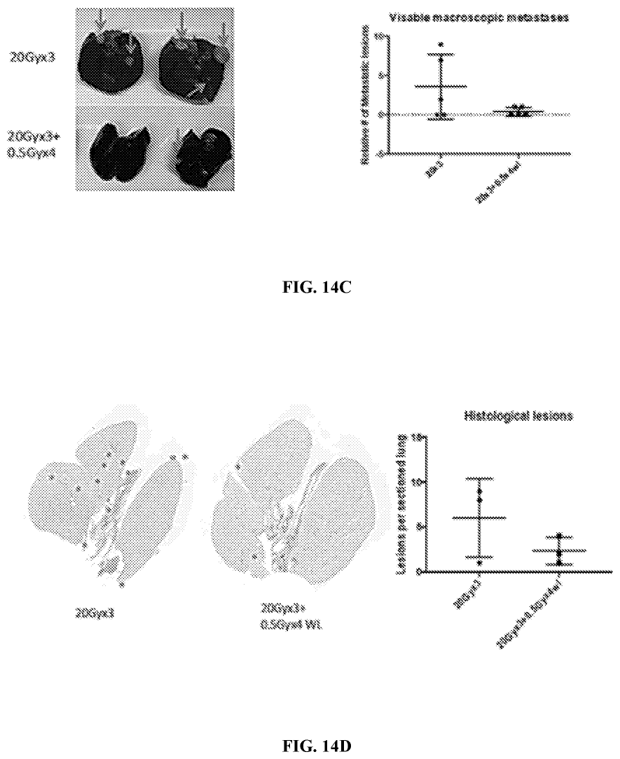

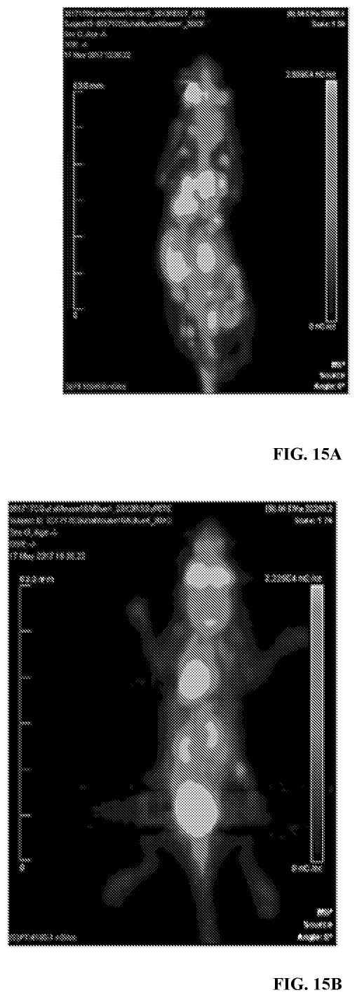

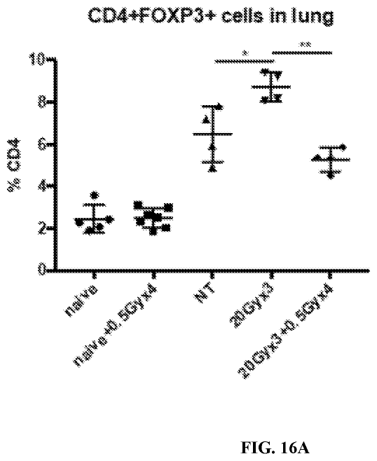

To investigate whether PAM-RT of metastases-prone organs can prevent progression after a course of hypofractionated ablative RT to the primary tumor in a poorly immunogenic, highly metastatic cancer course of local PAM-RT (22 Gy+0.5 Gy×4) was administered to orthotopic 4T1 breast cancer in Balb/c mice. Although primary tumor PAM-RT trended to delay local tumor progression, there was no increase in survival with all treated mice succumbing to metastatic disease (FIGS. 13A to C). Considering local treatment with PAM-RT remodeled the TME, the use of PAM-RT was translated to treat metastatic organs after primary tumor ablation. For adequate local tumor control, BalB/C mice with palpable 4T1 tumors were treated with three doses of 20 Gy delivered over 3 consecutive days to the primary tumor. As expected, primary tumor ablation alone could not rescue the animals from pulmonary metastases. Despite induction of an anti-tumoral immune response after primary tumor RT, RT-induced CTLs might be excluded from infiltration in metastatic sites. To test this the metastases-prone organ, the whole lung, was treated with daily doses of 0.5 Gy over 4 days, 12 days after completion of the primary tumor RT (FIG. 14A). Survival was significantly increased in these animals after whole lung PAM-RT, as compared to primary tumor RT alone, as shown in FIG. 14B. Investigation of metastatic burden of treated mice revealed fewer metastatic lesions in PAM-RT treated lungs by gross examination after India Ink injection, as seen in FIG. 14C, and in histologic specimens, FIG. 14D. PET scans of 4T1 mice showed similar results to the lung harvest, with lungs receiving PAM-RT having less metastatic burden (FIGS. 15A and B). These data indicate that PAM-RT doses of 0.5 Gy×4 can be administered either, directly to the primary tumor for local control or when treating systemic disease, delayed PAM-RT can be administered to the metastases-prone organ to slow tumor progression and increase survival.

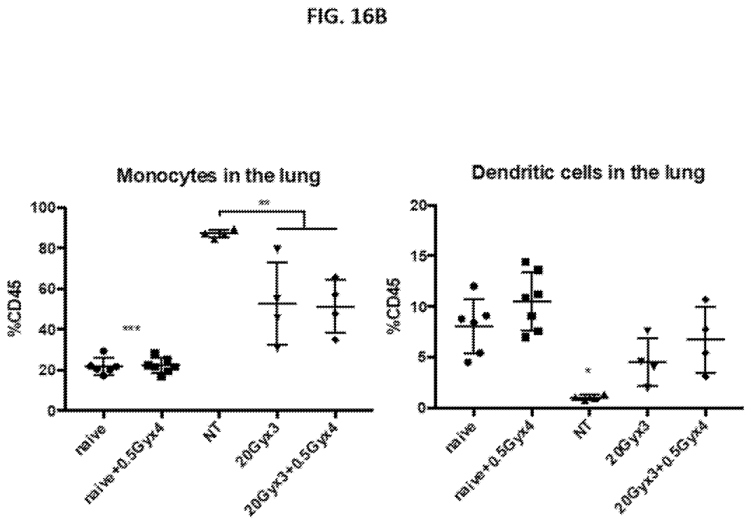

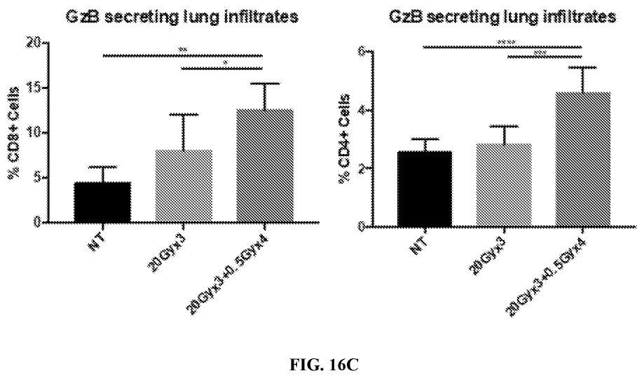

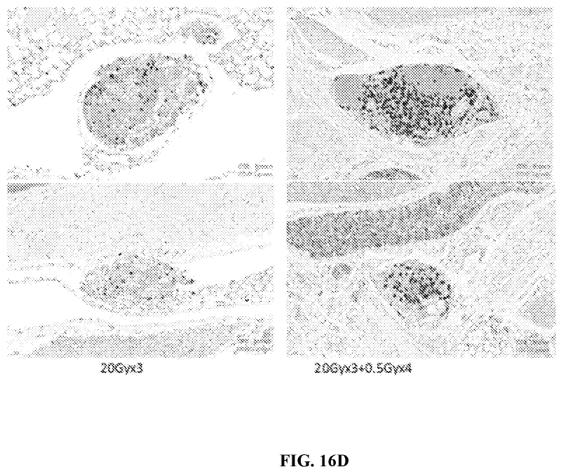

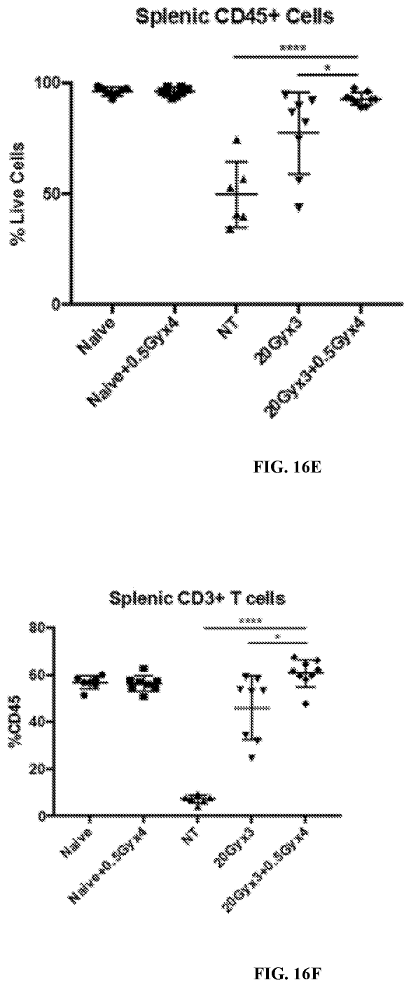

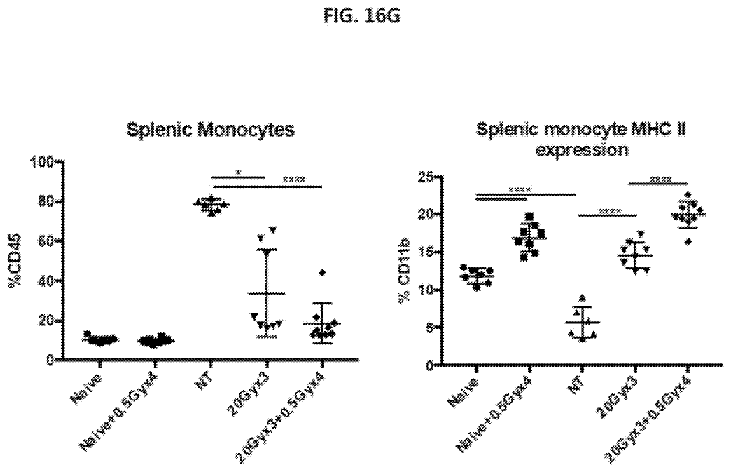

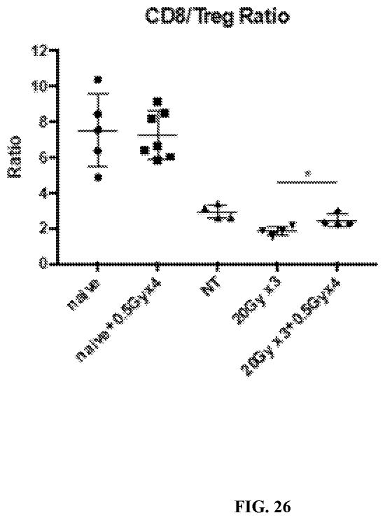

Systemic PAM-RT Remodels the Metastatic Niche with Decreased Tregs in Lungs
Characterization of immune cells in PAM-RT treated lungs showed similar results to local PAM-RT treatment, with a decrease in immunosuppressive phenotype of these cells. There was a significant decrease in Tregs, seen Figure in FIG. 16A, leading to a significantly increased CD8/Treg ratio in PAM-RT treated whole lungs (FIG. 26), FIG. 16B illustrates whole lung phenotyping by flow cytometry 19 days after primary tumor ablation Figure. Phenotyping of T cells in the lungs revealed significant increases in GzB secretion in both CD8+ and CD4+ T cells (FIG. 16C). Immunohistochemistry of micrometastases in the few metastatic lesions of PAM-RT treated lungs showed massive infiltration of CD8+ T cells, while FoxP3+ cells decreased, compared to lesions in mice receiving primary tumor ablation alone (FIG. 16D). In untreated animals, there were splenic metastases with enlargement of spleen and a reduction of CD45+ leucocytes. Upon primary tumor RT and primary tumor RT+lung PAM-RT splenic size decreased with subsequent increase in CD45+ leucocytes, FIG. 16E, and CD3+ T cell number, FIG. 16F, with RT+lung PAM-RT returning to baseline levels as seen in wild-type animals without tumors. A peripheral myeloid expansion is associated with G-CSF-secreting 4T1 tumors and in line with previous reports, there were massive increases in splenic macrophages amongst CD45+ leucocytes in mice with 4T1 tumors, with progressive reduction of these cells in mice treated with ablation alone versus ablation+PAM-RT, respectively. Interestingly, in animals with untreated tumor, splenic macrophages were immature with a reduced MHC Class II expression. Upon treatment with primary tumor ablation+/−lung PAM-RT, Class II MHC expression increased in macrophages significantly, indicating activation of macrophages with PAM-RT treatment (FIG. 16G). These experiments indicate that PAM-RT treatment can promote remodeling of the TME in the primary tumor, as well as, in metastatic site by reducing Tregs, activating macrophages to an inflammatory phenotype and promoting infiltration of CD8+ CTLs in metastatic tumors.

### Materials and Methods

### Cell Lines and Mice

The murine cell line Lewis Lung Carcinoma, 3LL, was purchased from the ATCC and grown in supplemented DMEM (10% FBS, 5% Sodium Pyruvate, 2.5% NEAA, 1% Anti-bacterial/Anti-mitotic). The murine breast carcinoma cell line, 4T1, was purchased from ATCC and grown in supplemented DMEM (10% FBS, 1% Anti-bacterial/Anti-mitotic). Cell lines were used between 4 and 8 passages and mycoplasma was tested every 4 months with MycoAlert (Lonza LT07-705). Six to eight week old C57BL/6 mice and ten to twelve week on BalB/C mice were ordered from NCI and athymic nude mice were ordered from Charles River. The Institutional Animal Care and Use Committee approved all studies performed.

### In Vivo Tumor Studies

C57BL/6 mice were challenged with 1.5×105 3LL cells subcutaneously and BalB/C mice were challenged orthotopically with 2×105 4T1 cells in the mammary fat pad. Tumors are allowed to grow until 5 mm in diameter before treatment. Tumor size was measured twice a week. Tumor volume was calculated using an ellipsoid formula: V=(π/6×length×width×height). CT guided radiation therapy of tumor-bearing mice

Radiation is delivered using Xstrahl's Limited Small Animal Radiation Research Platform(SARRP). Image guided radiation therapy is performed using the SARRPs on-board cone beam CT (CBCT). Following CBCT acquisition, the treatment plan is constructed using Muriplan.

### Tumor and Immune Cell Analysis

Tumor cells were cultured, plated, and treated with the specified radiation schemes. Cells were harvest 6 and 24 hours after the last treatment on day 5. Cells were harvested and stained for flow cytometry. Bone marrow derived macrophages were polarization to M1 (100 ng/mL LPS+50 ng/mL IFNg), M2 (10 ng/mL IL-4) and treated with radiation 24 hours later. T cell populations were sorted from spleens of naïve C57BL/6 mice with CD3, CD8, CD4, CD25 antibodies and allowed to rest overnight before radiation treatment. Immune cells were harvest 6 hours after final radiation dose.

### Tumor Processing

Tumors or whole lungs are harvested on ice, weighed and washed. After manual dissection with razor, the tumor or whole lung is transferred to 1 mL Digestion buffer (10% FBS, Collagenase I and IV at 100 u/mL(Sigma) and 1× DNase 1(Thermo Scientific)) in a 15 mL conical tube with a magnetic stir bar. Tubes are incubated at 37° C. for 15 min while rotating and transferred to a stir plate for 15 minutes manual digestion. Single cell suspensions are filtered. Cells are re-suspended for flow cytometry.

### Flow Cytometry Analysis

Cells were stained at 4° C. for 30 minutes with surface stain antibodies. Antibodies used included CD45, CD3, CD4, FOXP3, CD8, GzB, CD11b, WICK IL-10, TNFa and CD206. After washing, the cells are fixed with 4% PFA or permeabilized for intracellular staining BD Pharminogen Transcription Factor buffer set as per instructions. Intracellular stains are FOXP3, TNFa, IL-10 and Granzyme B, followed by fixation with 4% PFA. Cells are collected on the LSRII flow cytometer (BD Biosciences) and analyzed via Flow Jo software (Tree Star).

### Lymphoid Organ Harvest for Immune Analysis

Spleen and draining lymph nodes were harvested on ice and processed to single cell suspensions. In spleens, red blood cells were lysed with ACK lysis buffer (Lonza). Cells are counted before re-suspension in complete RPMI (10% heat-inactivated FBS, 1% Anti-biotic/Anti-mitotic). For intracellular cytokine analysis, Golgi-stop and monensin were added for 3 hours at 37° C. Single cell suspensions are further processed for flow cytometry analysis as done with tumor.

### ELISOPT Assay

Spleens processed for immune analysis were plated in coated ELISPOT plates at 1×10\hat{ }6 cells per well and incubated overnight before further processing. Reagents for IFNγ ELISPOTS were ordered from BD biosciences and manufacturer protocol followed. Regents for granzyme B ELISPOTs were ordered from R&D and manufacturer protocol followed.

### Histologic Staining of Tumor

Tumors and lungs were harvested, and transferred into 4% PFA or 10% formalin and stored at 4° C. Samples were then transferred to 70% and embedded in Paraffin. Blocks were sectioned and dual stained with CD8 and FOXP3.

### Statistical Analysis

Statistical analysis was performed using PRISM 7 (Graphpad Software) software. Analyses were performed using student's t test or ANOVA analysis with multiple group comparison. Data are representative mean±standard deviation. Survival curves were analyzed by Log Rank (Mantel-cox) and Grehan-Breslow-Wilcoxon tests. P values are represented as * p<0.05, ** p<0.005, *** p<0.0005, **** p<0.0001 for statistical significance.

### RT-PCR Analysis of TME

Six days after treatment start, tumors are harvested on ice and weighed. On ice, tumor is manually dissected and transferred to RNA later. For processing, samples are thawed on ice and Trizol is used to extract RNA as per manufacturers protocol. RNA is quantified on the Nanodrop with a 260 nm/280 nm ratio of around 2. cDNA synthesis is achieved using the Verso cDNA synthesis kit (Thermo Fisher) following the kit protocol. The Powerup SYBR green kit (Thermo Fisher) was used for the RT-PCR in a 384 well plate on an ABI 7900HT. Primers used are FOXP3 F-ACTCGCATGTTCGCCTACTTCAG and R-GGCGGATGGCATTCTTCCAGGT and β 2-microglobulin F-TTCTGGTGCTTGTCTCACTGA and R-CAGTATGTTCGGCTTCCCATTC. Delta CTs were calculated using SDS2.4 and compared against non treated controls.

### PET Imaging

Mice were fasted overnight and brought to the imaging facility. Mice were injected with 300-400 uCi (12-15 MBq) in 0.1 ml normal saline [18F] fluoro-2-deoxyglucose (FDG) via tail vein and imaging begins 1 hour after injection. Mice were anesthetized with isoflourane and imaged on an Inveon Multimodality scanner (Siemens). Analysis is performed using either ASIPRO and IRW (both Siemens) dedicated software.

### India Ink Treated Lungs

Treated 4T1 tumor bearing mice were sacrificed. Lungs were inflated with 10% India Ink by intra-tracheal injection. Lungs were harvested and washed in 1 L of water before storage in Feket's solution (300 ml 70% EtOH, 30 ml 37% formaldehyde, 5 ml glacial acetic acid) to allow for bleaching of macro-metastases. Metastatic lesions were enumerated by counting.

### Example 2 Quantitative and Qualitative T Cell Analysis after Radiation Treatment

To test the radiation treatment scheme, six-week-old C57BL/6 mice were inoculated with 3LL subcutaneously in the dorsum of the foot. Tumors were allowed to grow until 50 mm³ before treatment. Tumor size was measured three times a week for the duration of the study. Tumor volume was calculated using an ellipsoid formula: V=(π/6×length×width×height).

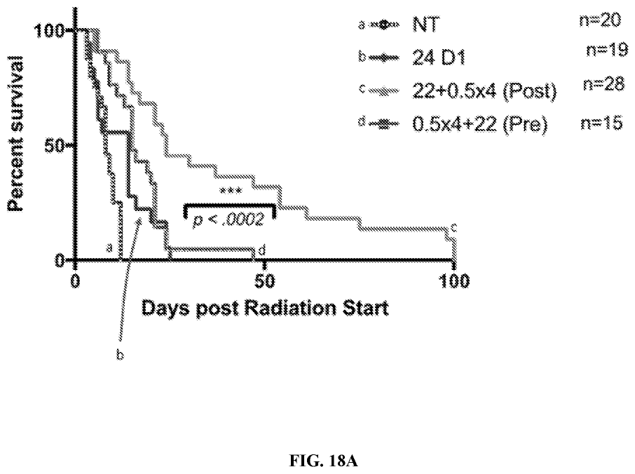

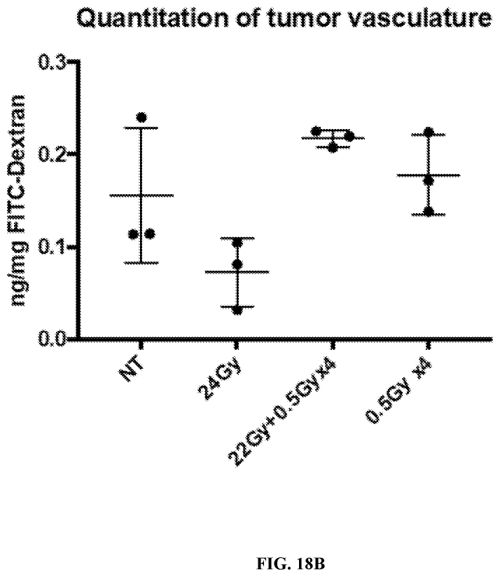

The inoculated mice were divided into four treatment groups. The first group received no treatment, the second group received a single dose of 24 Gy, the third group received a single dose of 22 Gy, followed 1 day later by four doses of 0.5 Gy, administered 1 day apart, and the fourth group received four doses of 0.5 Gy, administered 1 day apart, followed 1 day later by a single dose of 22 Gy. Tumor size was measured three times per week, and survival was assessed daily. As shown in FIG. 18A, the mice in the third group (c) had a better survival outcome than the mice in the first, second and fourth groups. Several mice from the third treatment group survived almost 3 months, while all the mice from the first and second groups, and most of the mice from the fourth group, were dead after approximately one month. A further group of mice were treated as above and injected with FITC-dextran 6 days after the first treatment to quantify tumor vasculature. As shown in FIG. 18B mice in the third and fourth groups exhibited increased vascular density compared to mice in the first and second groups.

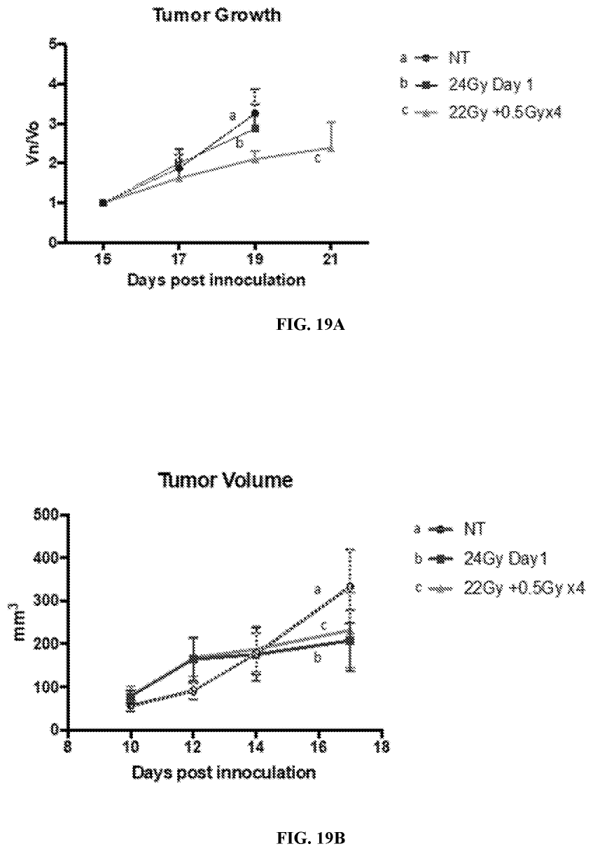

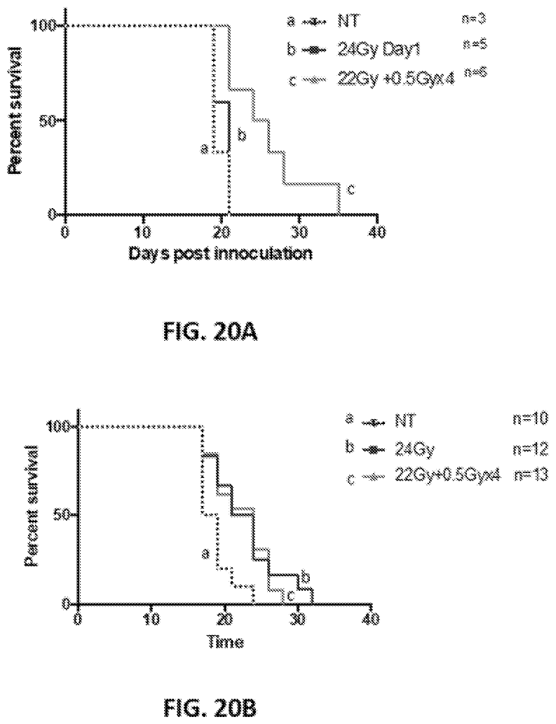

To investigate possible immune effects an additional experiment was performed using immunocompromised C57B16 mice and nude mice, which were split into three groups and treated like the first, second and third groups above. In the C57B16 mice tumor growth in the third group was slower than in the first and second groups, as seen in FIG. 19A. This effect was lost in the nude mice, as shown in FIG. 19B. Similarly, the survival benefit seen in the C57B16 mice was not observed in the nude mice, compare FIGS. 20A and 20B.

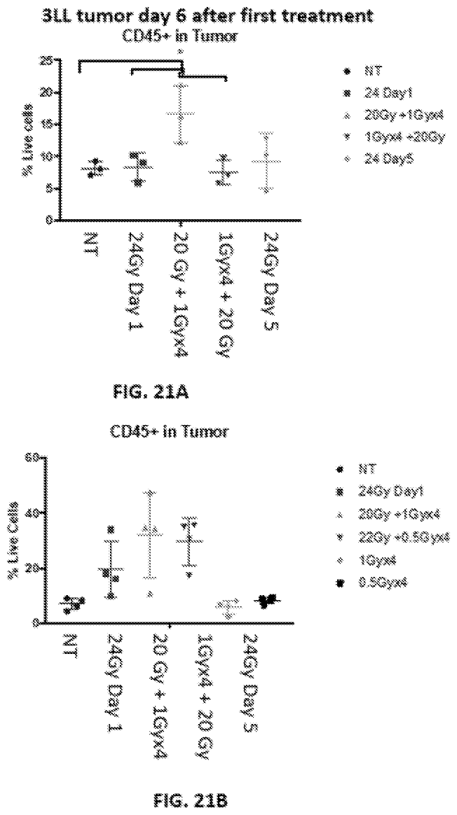

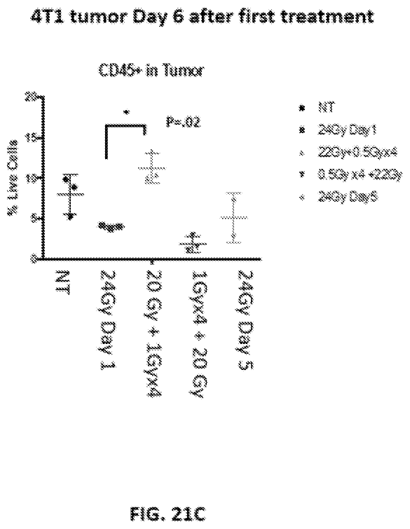

To investigate the effect of the radiation treatment on the immune response three different tumor cell lines were used: 1×10⁵ 3LL, 2×10⁵ UN-KC-6141, and 4×10⁵ MOE HPV E6/7/H-Ras cells. C57Bl6 mice were inoculated with the cells as above, and mice treated with each cell type were divided into six treatment groups: (1) no treatment, (2) 24 Gy, (3) 20 Gy followed 1 day later by four daily doses of 1 Gy, (4) 22 Gy followed 1 day later by four daily doses of 0.5 Gy, (5) four daily doses of 1 Gy, and (6) four daily doses of 0.5 Gy. Six days after the first treatment the tumors were harvested, and infiltration of leukocytes was analyzed by flow cytometry as described below. Increased infiltration of leukocytes was seen in group 3 compared to the other groups for all cell lines tested, see FIGS. 21A to C. Group 4 was more effective than groups 1, 2, 5 and 6 in the mice treated with the H-Ras cells (FIG. 21B), though not in the mice treated with the 3LL or 4T1 tumor cells.

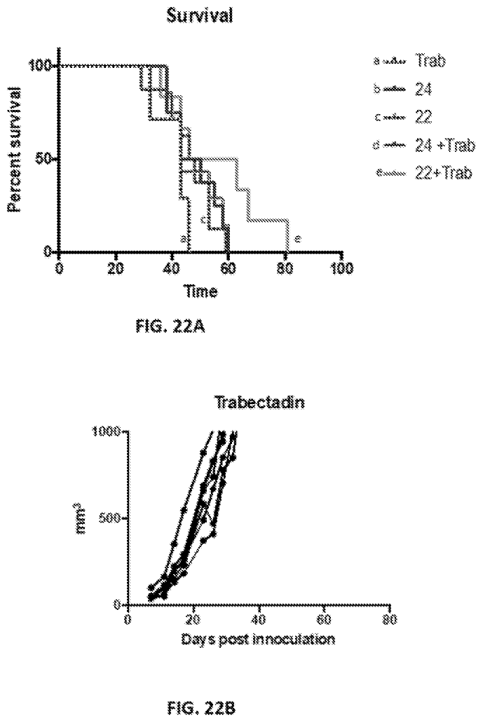

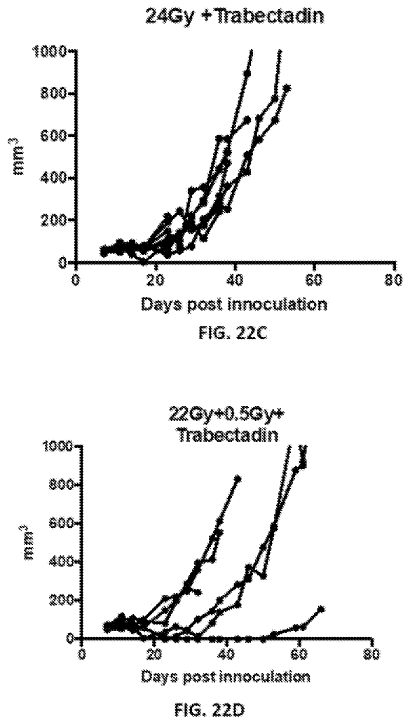

To determine whether the radiation schemes above are effective in combination therapies mice were injected with tumor cells as above and treated with Trabectadin alone, 24 Gy and Trabectedin or 22 Gy followed 1 day later with three weekly doses of 0.5 Gy and three weekly Trabectadin doses. Survival and tumor growth were recorded for each group. FIG. 22A shows the survival of mice in the different groups, showing highest survival rates in the group treated with or 22 Gy followed 1 day later with four daily doses of 0.5 Gy and Trabectadin. FIGS. 22B to D show the growth curves of the different tumors within each treatment group, the slowest growth rates are seen in the group which received or 22 Gy followed 1 day later with four daily doses of 0.5 Gy and Trabectadin (FIG. 22D).

A further combination experiment was performed with mice which were inoculated with 4T1 cells into the 4^{th} mammary fat pad. About eight days later the mice were treated as follows:

1. three doses of 20 Gy,

2. three doses of 20 Gy and an anti-PD1 therapeutic,

3. three doses of 20 Gy followed twelve days later by four daily doses of 0.5 Gy (administered to the whole lung), or

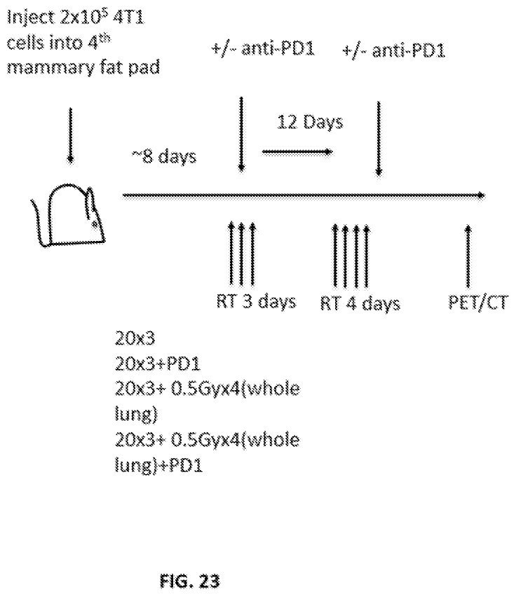

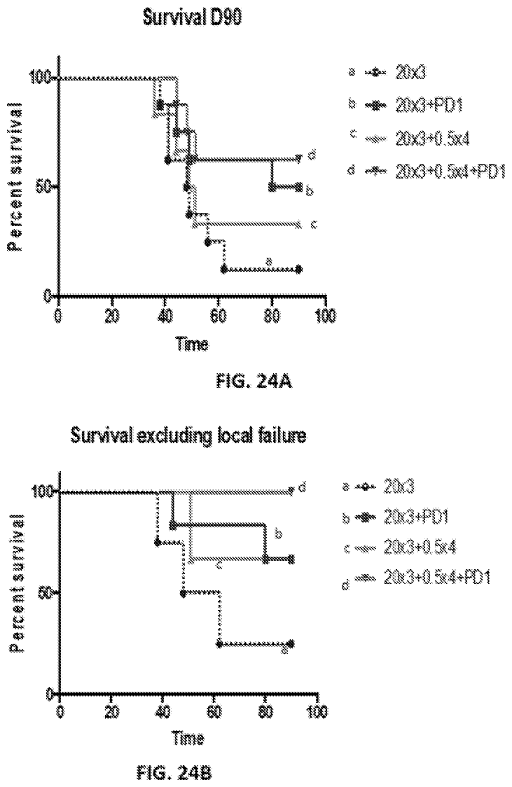

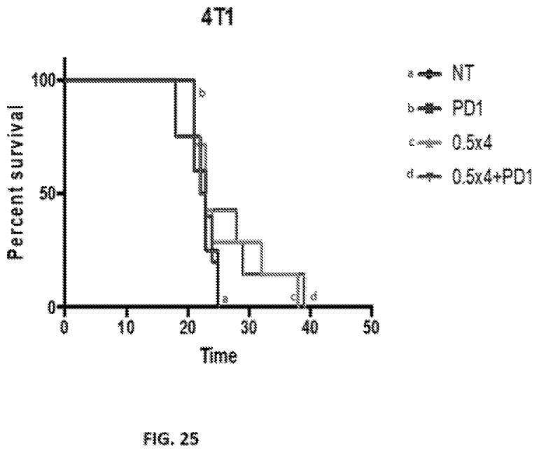

4. three doses of 20 Gy and an anti PD1 therapeutic, followed twelve days later by four daily doses of 0.5 Gy (administered to the whole lung) and an anti PD1 therapeutic. The treatment regime is illustrated in FIG. 23. As shown in FIGS. 24A and 24B (excluded the mice with local failure of the primary tumor, possibly confounding survival, the mice treated in group 4 showed the longest survival. Mice administered 4T1 cells intravenously and treated with either an anti PD1 therapeutic alone, four daily doses of 0.5 Gy alone, or the anti-PD1 therapeutic and four daily doses of 0.5 Gy had similar survival rates to untreated mice, see FIG. 25.

### Methods

### Cell Lines and Mice

The murine cell line Lewis Lung Carcinoma, 3LL, was purchased from the ATCC and grown in supplemented DMEM (10% FBS, 5% Sodium Pyruvate, 2.5% NEAA, 1% Pen/Strep). The 4T1 Murine Breast Carcinoma was purchased from ATCC and grown in supplemented DMEM (10% FBS, 1% Pen/Strep)

Six-week old C57BL/6 mice were ordered from NCI and maintained under pathogen-free conditions.

### CT Guided Radiation Therapy of Tumor-Bearing Mice

Radiation is delivered using Xstrahl's Limited Small Animal Radiation Research Platform(SARRP). Image guided radiation therapy is performed using the SARRPs on-board cone beam CT (CBCT). Following CBCT acquisition, the treatment plan is constructed using Muriplan.

### Immunotherapeutic Treatment of Tumor-Bearing Mice

Tumor bearing mice randomized to the immunotherapy treatment groups received 200 ug PD-1 i.p. every three days for a total of 5 doses, αCD40, 4-1BBL or GM-CSF, with or without antigenic vaccination.

### Syntac Administration

Syntac constructs conjugating a T cell co-stimulatory domain 41BBL to antigen specific for the tumor model will be used to induce activated antigen specific effector CD8+ T cells. Tumor bearing mice randomized to syntac receive one 300 ug i.p. treatment. Mice receiving antigen specific syntac treatment are compared to mice treated with an irrelevant syntac or the co-stimulatory domain 4-1BBL alone.

### Tumor Digestion

Tumors were harvested on ice and weighed. After manual dissection with razor blades into 1 mm×1 mm pieces, the tumors were transferred to 1 mL Digestion buffer, 5% FBS, Collagenase I and IV at 100 u/mL(Sigma) and 1× DNase 1(Thermo Scientific), in a 15 mL conical tube with a magnetic stir bar. Tubes were incubated at 37° C. for 30 min and transferred to a stir plate for 30 minutes manual digestion. Single cell suspensions were filtered and re-suspended for analysis.

### Flow Cytometry Analysis of Immune Cells

Single cell suspension cells were stained at 4° C. for 30 minutes with surface stain antibodies. After washing, the cells were fixed with 4% PFA or permeabilized for intracellular staining. BD Pharminogen Transcription Factor buffer set as per instructions. Intracellular stains were FOXP3 and Ki-67 followed by fixation with 4% PFA. Cells were collected on the LSRII flow cytometer (BD Biosciences) and analyzed via Flow Jo software (Tree Star).

### Antibodies

Immunoflourescence antibodies: Primary antibodies used were CD3, CD8, CD4, CD11b, Gr1 (BD Pharminogen). Secondary antibodies were goat anti-rabbit FITC and goat anti-rat APC (Abcam).

Flow cytometry antibodies: CD45, CD3, CD4, CD8, CD69, PD1, CD11b, Gr1, Ly6C, LY6G, CD11c, NK1.1, NKG2D, MEW II (IA) and F4/80.

### Tumor Microenvironment Analysis

Six days after treatment, tumors were harvested on ice and weighed. Being kept on ice, tumors were transferred to 1.7 mL Eppendorf tubes and were homogenized in 4.5 uL PBS with 1× Protease and phosphatase inhibitor(Cell signaling) per milligram of tissue. Tubes were spun down at 14,000 g for 15 minutes. Supernatants were transferred to a clean tube and stored at −80° C.

Vascular Density Quantification with FITC Dextran

Mice were injected with 1×10\hat{ }5 3LL subcutaneously in the flank. Tumors were allowed to for 14 days to about 6 mm. Mice were randomized to no treatment, 24 Gy day 1, 22 Gy+0.5 Gy×4, or 0.5 Gy×4 starting on day 2. Six days after start of treatment, 70 kDa FITC Dextran(25 mg/mL) was injected intravenously and allowed to circulate for 20 min. Mice were then sacrificed and blood and tumor harvested. Tumors were weighed and incubated overnight with 1.5 mL Collagenase IV at 37 C. Tumors were then homogenized and cell debris spun down at 14000 rpm for 5 min. Supernatants were collected and run at 485/535 for fluorescence. Fluorescence per mg was then calculated. Blood sera was used as an injection control.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a cancer in a subject, the method comprising: providing an ablative dose of radiation therapy to a first region comprising the cancer followed by a sub-ablative dose to a second region, wherein the sub-ablative dose is administered after the ablative dose.

2. The method of claim 1, wherein the sub-ablative dose is administered at least 1 hour after the ablative dose.

3. The method of claim 1, wherein the sub-ablative dose is administered at least 1 day after the ablative dose.

4. The method of claim 1, wherein the sub-ablative dose is administered no more than 4 days after the ablative dose.

5. The method of claim 1, wherein a cumulative amount of radio therapy delivered to the second region throughout a course of treatment comprises less than an ablative dose.

6. The method of claim 5 wherein the cumulative amount less than an ablative dose comprises a plurality of sub-ablative doses.

7. The method of claim 1, wherein the first region comprises a region of a tumor and optionally wherein the second region comprises the region of the tumor.

8. The method of claim 1, wherein the first region comprises a region of a first tumor and the second region comprises a region of a second tumor.

9. The method of claim 8 wherein the first tumor comprises a primary tumor and the second tumor comprises a metastatic tumor.

10. The method of claim 8 wherein the first tumor comprises a metastatic tumor and the second tumor comprises a primary tumor.

11. The method of claim 8 wherein the second region comprises a plurality of second regions and wherein each of the plurality of second regions receives a cumulative amount of radiotherapy which is less than an ablative dose.

12. The method of claim 1, wherein the second region comprises a different region from the first region.

13. The method of claim 1, wherein the second region comprises a region of a tumor.

14. The method of claim 1, wherein the second region comprises a region likely to develop a metastatic tumor and optionally wherein the second region comprises a region of an organ selected from the group consisting of bones, lymph node, lung, liver, brain, adrenal gland, breast, eye, kidney, muscles, pancreas, salivary gland, and spleen.

15. The method of claim 1, wherein the second region comprises a whole body of the subject scanned with the sub-ablative dose.

16. The method of claim 1, wherein the first region comprises a region of a primary tumor of an organ selected from the group consisting of breast, bladder, brain, colon, rectal, endometrial, kidney, pancreas, prostate, liver, lung, skin, thyroid, uterus, lymph node, tonsil, thymus, spleen and bone marrow and the second region comprises a region of a metastatic tumor of an organ selected from the group consisting of bones, lymph node, lung, liver, brain, adrenal gland, breast, eye, kidney, muscles, pancreas, salivary gland, and spleen.

17. The method of claim 1, wherein the first region comprises a region of a metastatic tumor of an organ selected from the group consisting of bone, lymph node, lung, liver, brain, adrenal gland, breast, eye, kidney, muscles, pancreas, salivary gland and spleen, and the second region comprises a primary tumor of an organ selected from the group consisting of breast, bladder, brain, colon, rectal, endometrial, kidney, pancreas, prostate, liver, lung, skin, thyroid, uterus, lymph node, tonsil, thymus, spleen and bone marrow.

18. The method of claim 1, wherein the first region comprises an identified tumor and the second region does not comprise an identified tumor.

19. A computer readable medium configured with instructions that, when executed, cause a processor to: provide instructions to a radiotherapy system to deliver an ablative dose of radiation therapy to a first region followed by delivery of a sub-ablative dose to a second region after the ablative dose.

20. The computer readable medium of claim 19, wherein the sub-ablative dose is delivered at least 1 hour after the ablative dose.

21. The computer readable medium of claim 19, wherein the sub-ablative dose is delivered at least 1 day after the ablative dose.

22. The computer readable medium of claim 19, wherein the sub-ablative dose is delivered no more than 4 days after the ablative dose.

23. The computer readable medium of claim 19, wherein a cumulative amount of radio therapy delivered to the second region throughout a course of treatment comprises an amount of radio therapy which is less than an ablative dose.

24. The computer readable medium of claim 23, wherein the cumulative amount of radio therapy less than an ablative dose comprises a plurality of sub-ablative doses.

25. The computer readable medium of claim 19, wherein the first region comprises a region of a tumor and optionally wherein the second region comprises the region of the tumor.

26. The computer readable medium of claim 19, wherein the first region comprises a region of a first tumor and the second region comprises a region of a second tumor

27. The computer readable medium of claim 26, wherein the first tumor comprises a primary tumor and the second tumor comprises a metastatic tumor.

28. The computer readable medium of claim 26, wherein the first tumor comprises a metastatic tumor and the second tumor comprises a primary tumor.

29. The computer readable medium of claim 26, wherein the second region comprises a plurality of second regions and wherein each of the plurality of second regions receives a cumulative amount of radiotherapy which is less than an ablative dose.

30. The computer readable medium of claim 19, wherein the second region comprises a different region from the first region.

31. The computer readable medium of claim 19, wherein the second region comprises a region of a tumor.

32. The computer readable medium of claim 19, wherein the second region comprises a region likely to develop a metastatic tumor and optionally wherein the second region comprises a region of an organ selected from the group consisting of bones, lymph node, lung, liver, brain, adrenal gland, breast, eye, kidney, muscles, pancreas, salivary gland, and spleen.

33. The computer readable medium of claim 19, wherein the second region comprises a whole body of the subject scanned with the sub-ablative dose.

34. The computer readable medium of claim 19, wherein the first region comprises a region of a primary tumor of an organ selected from the group consisting of breast, bladder, brain, colon, rectal, endometrial, kidney, pancreas, prostate, liver, lung, skin, thyroid, uterus, lymph node, tonsil, thymus, spleen and bone marrow and the second region comprises a region of a metastatic tumor of an organ selected from the group consisting of bones, lymph node, lung, liver, brain, adrenal gland, breast, eye, kidney, muscles, pancreas, salivary gland, and spleen.

35. The computer readable medium of claim 19, wherein the first region comprises a region of a metastatic tumor of an organ selected from the group consisting of bone, lymph node, lung, liver, brain, adrenal gland, breast, eye, kidney, muscles, pancreas, salivary gland and spleen, and the second region comprises a primary tumor of an organ selected from the group consisting of breast, bladder, brain, colon, rectal, endometrial, kidney, pancreas, prostate, liver, lung, skin, thyroid, uterus, lymph node, tonsil, thymus, spleen and bone marrow.

36. The computer readable medium of claim 19, wherein the first region comprises an identified tumor and the second region does not comprise an identified tumor.

37. A radiotherapy system comprising:
a source of radiation to provide an ablative dose and a sub-ablative dose; and
a processor coupled to the source of radiation, wherein the processor is configured with the instructions of any one of claims 19 to 36.

38. The method, system, or computer readable medium of any one of claims 1 to 37, wherein the ablative dose comprises between 20 and 100 Gy at the first region.

39. The method, system, or computer readable medium of any one of claims 1 to 37, wherein the ablative dose comprises between 20 and 60 Gy at the first region.

40. The method, system, or computer readable medium of any one of claims 1 to 37, wherein the sub-ablative dose comprises between 0.1 and 2 Gy and optionally wherein the sub-ablative dose comprises a plurality of sub-ablative doses and each of the plurality of sub-ablative doses comprises between 0.1 and 2 Gy at the second region.

41. The method, system, or computer readable medium of any one of claims 1 to 37, wherein the sub-ablative dose comprises between 0.1 and 0.5 Gy and optionally wherein the sub-ablative dose comprises a plurality of sub-ablative doses and each of the plurality of sub-ablative dose/s comprises between 0.1 and 5 Gy at the second region.

42. The method, system, or computer readable medium of any one of claims 1 to 41, wherein three sub-ablative doses are administered.

43. The method, system, or computer readable medium of any one of claims 1 to 41, wherein more than three sub-ablative doses are administered.

44. The method, system, or computer readable medium of any one of claims 1 to 41, wherein a first sub-ablative dose is administered within 24 hours after the administration of the ablative dose.

45. The method, system, or computer readable medium of any one of claims 1 to 41, wherein a first sub-ablative dose is administered between 6 and 26 hours after the administration of the ablative dose.

46. The method, system, or computer readable medium of any one of claims 1 to 45, wherein the treatment reduces the size or intensity of the treated tumor as measured by imaging selected from the group consisting of a computed tomography scan, magnetic resonance imaging, positron emission tomography, a computed tomography scan.

47. The method, system, or computer readable medium of any one of claims 1 to 45, wherein the treatment increases the survival of the subject, reduces the number or severity of symptoms experienced by the subject, increases a number of immune cells in a microenvironment of the tumor, or increases a number of activated immune cells in a tumor microenvironment.

48. The method, system, or computer readable medium of any one of claims 1 to 45, wherein the radiation is selected from the group consisting of x-ray radiation, gamma ray radiation, alpha particle radiation, beta particle radiation, neutron particle radiation, external beam radiation and brachytherapy.
